(19) United States
(12) Patent Application Publication
(10) Pub. No.: US 2003/0144506 A1
(43) Pub. Date: Jul. 31, 2003

(54) Processes for the preparation of substituted bicyclic derivatives for the treatment of abnormal cell growth

(75) Inventor: David Harold Brown, Westbrook, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/307,603

(22) Filed: Dec. 2, 2002

Related U.S. Application Data
(60) Provisional application No. 60/334,647, filed on Nov. 30, 2001.

(51) Int. Cl. C07D 417/02; C07D 413/02; C 07D 4 3/02; C07F 15/00; C07D 239/72

(52) U.S. Cl. 544/60; 544/284; 544/238; 544/116; 544/293; 556/18

(57) ABSTRACT

The invention relates to processes for preparing compounds of the formula 1

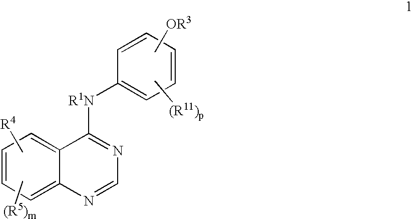

and to pharmaceutically acceptable salts, prodrugs and solvates thereof, wherein R¹, R³, R⁴, R⁵, R¹¹, m and p are as defined herein. The compounds of formula 1 are useful in treating abnormal cell growth in mammals by administering pharmaceutical compositions.

This application claims the benefit of U.S. Provisional Application Serial No. 60/34,647, filed Nov. 30, 2001 the contents of which are hereby incorporated by reference.

### BACKGROUND OF THE INVENTION

This invention relates to processes for the preparation compounds of formula 1

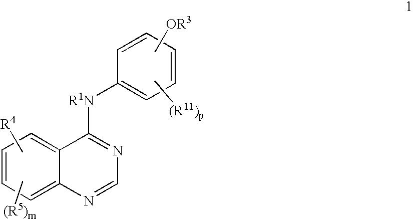

and to pharmaceutically acceptable salts, solvates and prodrugs thereof wherein R¹, R³, R⁴, R⁵, R¹¹, m and p are as defined herein.

The compounds of formula 1 are useful in the treatment of abnormal cell growth, such as cancer, in mammals, are described in U.S. patent application Ser. No. 09/883,752, filed Jun. 18, 2001, the contents of which are hereby incorporated by reference.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Other receptor tyrosine kinases include c-erbB-2, c-met, tie-2, PDGFr, FGFr, and VEGFR. It is known that such kinases are frequently aberrantly expressed in common human cancers such as breast cancer, gastrointestinal cancer such as colon, rectal or stomach cancer, leukemia, and ovarian, bronchial or pancreatic cancer. It has also been shown that epidermal growth factor receptor (EGFR), which possesses tyrosine kinase activity, is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma which expresses epidermal growth factor receptor tyrosine kinase (EGFR) but is without effect on the growth of another carcinoma which does not express the EGF receptor. Thus, the compounds of the present invention, which are selective inhibitors of certain receptor tyrosine kinases, are useful in the treatment of abnormal cell growth, in particular cancer, in mammals. In addition to receptor tyrosine kianses, the compounds of the present invention can also display inhibitory activity against a variety of other non-receptor tyrosine kinases (eg: lck, src, abl) or serine/threonine kinases (e.g.: cyclin dependent kinases).

Various other compounds, such as styrene derivatives, have also been shown to possess tyrosine kinase inhibitory properties. Five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are U.S. patent application Ser. Nos. 09/488,350 (filed Jan. 20, 2000) and 09/488,378 (filed Jan. 20, 2000), both of which are incorporated herein by reference in their entirety.

### SUMMARY OF THE INVENTION

The present invention relates to a process for preparing a compound of formula 1

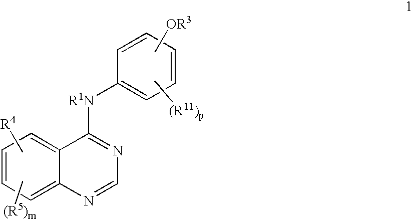

pharmaceutically acceptable salts, solvates and prodrugs thereof, wherein:

m is an integer from 0 to 3;

p is an integer from 0 to 4;

each R¹ and R² is independently selected from H and C₁-C₆ alkyl;

R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;

R⁴ is —C≡C—(CR¹⁶R¹⁷)ₜR⁹, —C═C—(CR¹⁶R¹⁷)ₜ—R⁹, —C≡C—(CR¹⁶R¹⁷)ₖR¹³, or —C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3;

each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;

each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;

or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;

each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R² )ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;

R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;

each R¹¹ is independently selected from the substituents provided in the definition of R⁸, except R¹¹ is not oxo(═O);

R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;

R¹³ is —NR¹R¹⁴ or —OR¹⁴;

R¹⁴ is H, R¹⁵, —C(O)R¹⁵, —SO₂R¹⁵, —C(O)NR¹⁵R⁷, —SO₂NR¹⁵R⁷, or —CO₂R¹⁵;

R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;

each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

R¹⁸ is C₁-C₆ alkyl wherein each carbon not bound to a N or O atom, or to S(O)ⱼ, wherein j is an integer from 0 to 2, is optionally substituted with R¹²;

and wherein any of the above-mentioned substituents comprising a CH₃ (methyl), CH₂ (methylene), or CH (methine) group, which is not attached to a halogeno, SO or SO₂ group or to a N, O or S atom, is optionally subsituted with a group selected from hydroxy, halo, C₁-C₄ alkyl, C₁-C₄ alkoxy and —NR¹R², which comprises reacting a compound of formula 2

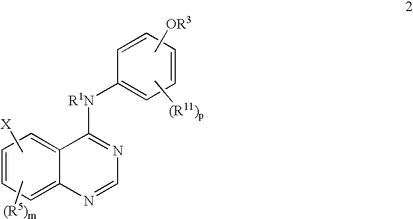

wherein X is a halide and R¹, R³, R⁵, R¹¹, m and p are as defined for formula 1 with a compound of formula H—C≡C—(CR¹⁶R¹⁷)ₜR⁹, M-C═C—(CR¹⁶R¹⁷)ₜ—R⁹, H—C≡C—(CR¹⁶R¹⁷)ₖR¹³, or M-C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3, wherein M is selected from the group consisting of H, B(R¹⁹)₂, Al(R²⁰)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo, wherein R²⁰ is selected from the group consisting of C₁-C₁₀ alkyl, C₁-C₁₀ alkoxy, C₃-C₁₀ cycloalkyl, and halo, wherein R²¹ is C₁-C₁₀ alkyl and wherein W is Cl, Br or I, wherein said reaction is carried out in the presence of a palladium catalyst, a ligand, a base, and an optional additive.

The present invention also relates to a second process for the preparing a compound of formula 1

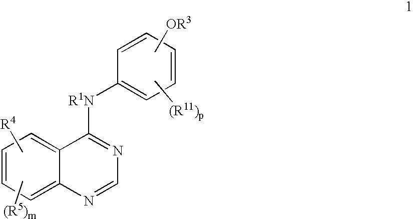

pharmaceutically acceptable salts, solvates and prodrugs thereof, wherein:

m is an integer from 0 to 3;

p is an integer from 0 to 4;

each R¹ and R² is independently selected from H and C₁-C₆ alkyl;

R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;

R⁴ is —C≡C—(CR¹⁶R¹⁷)ₜR⁹, —C═C—(CR¹⁶R¹⁷)ₜ—R⁹, —C≡C—(CR¹⁶R¹⁷)ₖR¹³, or —C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3;

each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;

each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;

or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;

each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;

R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;

each R¹¹ is independently selected from the substituents provided in the definition of R⁸, except R¹¹ is not oxo(═O);

R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;

R¹³ is —NR¹R¹⁴ or —OR¹⁴;

R¹⁴ is H, R¹⁵, —C(O)R¹⁵, —SO₂R¹⁵, —C(O)NR¹⁵R⁷, —SO₂NR¹⁵R⁷, or —CO₂R¹⁵;

R¹⁵is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;

each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

R¹⁸ is C₁-C₆ alkyl wherein each carbon not bound to a N or O atom, or to S(O)ⱼ, wherein j is an integer from 0 to 2, is optionally substituted with R¹²;

and wherein any of the above-mentioned substituents comprising a CH₃ (methyl), CH₂ (methylene), or CH (methine) group, which is not attached to a halogeno, SO or SO₂ group or to a N, O or S atom, is optionally subsituted with a group selected from hydroxy, halo, C₁-C₄ alkyl, C₁-C₄ alkoxy and —NR¹R², which comprises reacting a compound of formula 3

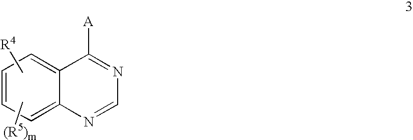

wherein A is Cl or F and R⁴, R⁵ and m are as defined for formula 1 with a compound of formula 4

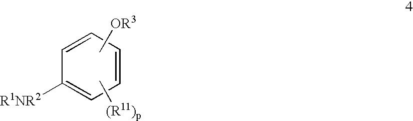

wherein R¹, R², R³, R¹¹ and p are as defined for formula 1.

In a specific embodiment of the present invention the processes of the present invention are used to make compounds of formula 1 wherein R³ is —(CR¹R²)ₜ(4 to 10 membered heterocyclic), wherein t is an integer from 0 to 5, and the foregoing R³ groups are optionally substituted by 1 to 3 R⁸ groups; said heterocyclic group is optionally fused to a benzene ring or a C₅-C₈ cycloalkyl group, and the foregoing R³ groups, including any optional fused rings referred to above, are optionally substituted by 1 to 3 R⁸ groups.

Other specific embodiments of the present invention the processes of the present invention are used to make compounds of formula 1, wherein R³ is selected from

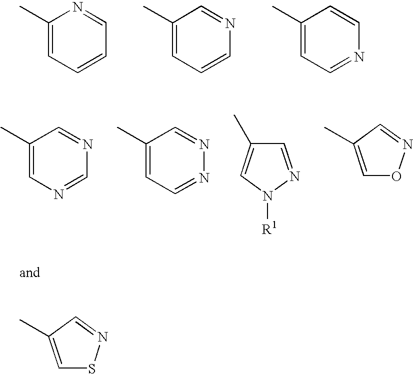

wherein the foregoing R³ groups are optionally substituted by 1 to 3 R⁸ groups.

Other specific embodiments the processes of the present invention are used to make compounds of formula 1 wherein R³ is pyridin-3-yl optionally substituted by 1 to 3 R⁸ groups.

Other specific embodiments the processes of the present invention are used to make compounds of formula 1, wherein R⁴ is —C≡C—(CR¹⁶R¹⁷)ₜR⁹, wherein m is an integer from 0 to 3, and t is an integer from 0 to 5.

Other specific embodiments the processes of the present invention are used to make compounds of formula 1, wherein R⁴ is —C≡C—(CR¹⁶R¹⁷)ₜR⁹, wherein m is an integer from 0 to 3, and t is an integer from 0 to 5, wherein R⁹ is selected from 3-piperidinyl and 4-piperidinyl each of which is optionally substituted with I or 2 R⁸ groups.

Other specific embodiments the processes of the present invention are used to make compounds of formula 1, wherein R⁴is —C═C—(CR¹⁶R¹⁷)ₜ—R⁹, wherein m is an integer from 0 to 3, and t is an integer from 0 to 5.

Other specific embodiments the processes of the present invention are used to make compounds of formula 1, wherein R⁴is —C═C—(CR¹⁶R¹⁷)ₜ—R⁹, wherein m is an integer from 0 to 3, and t is an integer from 0 to 5, wherein R⁹ is selected from 3-piperidinyl and 4-piperidinyl (optionally substituted with 1 or 2 R⁸ groups).

Other specific embodiments the processes of the present invention are used to make compounds of formula 1, wherein R⁴ is —C≡C—(CR¹⁶R¹⁷)ₖR¹³, wherein k is an integer from 1 to 3 and m is an integer from 0 to 3.

Other specific embodiments the processes of the present invention are used to make compounds of formula 1, wherein R⁴is —C≡C—(CR¹⁶R¹⁷)ₖR¹³, wherein k is an integer from 1 to 3 and m is an integer from 0 to 3, wherein R¹³ is —NR¹R¹⁴, wherein R¹⁴ is selected from —C(O)R¹⁵, —SO₂R¹⁵, and —C(O)NR¹⁵R⁷.

Other specific embodiments the processes of the present invention are used to make compounds of formula 1, wherein R⁴ is —C═C—(CR¹⁶R¹⁷)ₖR¹³, wherein k is an integer from 1 to 3 and m is an integer from 0 to 3.

Other specific embodiments the processes of the present invention are used to make compounds of formula 1, wherein R⁴ is —C═C—(CR¹⁶R¹⁷)ₖR¹³, wherein k is an integer from 1 to 3 and m is an integer from 0 to 3, wherein R¹³ is —NR¹R¹⁴, wherein R¹⁴ is selected from —C(O)R¹⁵, —SO₂R¹⁵, and —C(O)NR¹⁵R⁷.

Other specific embodiments of the processes of the present invention are used to make compounds of formula 1, wherein R⁴ is —C≡C—(CR¹⁶R¹⁷)ₖR¹³ or —C═C—(CR¹⁶R¹⁷)ₖR¹³, wherein k is an integer from 1 to 3 and m is an integer from 0 to 3, R¹³ is —NR¹R¹⁴ or —OR¹⁴, R¹⁴ is R¹⁵, R¹⁵ is R¹⁸, and R¹⁸ is C₁-C₆ alkyl optionally substituted by —OR⁶, —S(O)ⱼR⁶, —NR⁶R⁷, —NR⁶C(O)R⁷, —NR⁶SO₂R⁷, —NR⁶CO₂R⁷, CN, —C(O)R⁶, or halo.

Specific preferred compounds prepared using the processes of the present invention include those selected from the group consisting of:

(±)-[3-Methyl-4-(pyridin-3-yloxy)-phenyl]-(6-piperidin-3-ylethynyl-quinazolin-4-yl)-amine;

2-Methoxy-N-(3-{4-[3-methyl-4-(pyridin-3-yloxy)-phenylamino]-quinazolin-6-yl}-prop-2-ynyl)-acetamide

(±)-[3-Methyl-4-(6-methyl-pyridin-3-yloxy)-phenyl]-(6-piperidin-3-ylethynyl-quinazolin-4-yl)-amine;

[3-Methyl-4-(6-methyl-pyridin-3-yloxy)-phenyl]-(6-piperidin-4-ylethynyl-quinazolin-4-yl)-amine;

2-Methoxy-N-(3-{4-[3-methyl-4-(6-methyl-pyridin-3-yloxy)-phenylamino]-quinazolin-6-yl}-prop-2-ynyl)-acetamide;

2-Fluoro-N-(3-{4-[3-methyl-4-(6-methyl-pyridin-3-yloxy)-phenylamino]-quinazolin-6-yl}-prop-2-ynyl)-acetamide;

E-2-Methoxy-N-(3-{4-[3-methyl-4-(6-methyl-pyridin-3-yloxy)-phenylamino]-quinazolin-6-yl}-allyl)-acetamide;

[3-Methyl-4-(pyridin-3-yloxy)-phenyl]-(6-piperidin-4-ylethynyl-quinazolin-4-yl)-amine;

2-Methoxy-N-(1-{4-[3-methyl-4-(6-methyl-pyridin-3-yloxy)-phenylamino]-quinazolin-6-ylethynyl}-cyclopropyl)-acetamide;

E-N-(3-{4-[3-Chloro-4-(6-methyl-pyridin-3-yloxy)-phenylamino]-quinazolin-6-yl}-allyl)-2-methoxy-acetamide;

N-(3-{4-[3-Chloro-4-(6-methyl-pyridin-3-yloxy)-phenylamino]-quinazolin-6-yl}-prop-2-ynyl)-acetamide;

N-(3-{4-[3-Methyl-4-(6-methyl-pyridin-3-yloxy)-phenylamino]-quinazolin-6-yl}-prop-2-ynyl)-acetamide;

E-N-(3-{4-[3-Chloro-4-(6-methyl-pyridin-3-yloxy)-phenylamino]-quinazolin-6-yl}-allyl)-acetamide;

E-2-Ethoxy-N-(3-{4-[3-methyl-4-(6-methyl-pyridin-3-yloxy)-phenylamino]-quinazolin-6-yl}-allyl)-acetamide;

1-Ethyl-3-(3-{4-[3-methyl-4-(6-methyl-pyridin-3-yloxy)-phenylamino]-quinazolin-6-yl}-prop-2-ynyl)-urea;

Piperazine-1-carboxylic acid (3-{4-[3-methyl-4-(6-methyl-pyridin-3-yloxy)-phenylamino]-quinazolin-6-yl}-prop-2-ynyl)-amide;

(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;

2-Dimethylamino-N-(3-{4-[3-methyl-4-(pyridin-3-yloxy)-phenylamino]-quinazolin-6-yl}-prop-2-ynyl)-acetamide;

E-N-(3-{4-[3-Methyl-4-(6-methyl-pyridin-3-yloxy)-phenylamino]-quinazolin-6-yl}-allyl)-methanesulfonamide;

Isoxazole-5-carboxylic acid (3-{4-[3-methyl-4-(6-methyl-pyridin-3-yloxy)-phenylamino]-quinazolin-6-yl}-prop-2-ynyl)-amide;

1-(1,1-Dimethyl-3-{4-[3-methyl-4-(6-methyl-pyridin-3-yloxy)-phenylamino]-quinazolin-6-yl}-prop-2-ynyl)-3-ethyl-urea;

and the pharmaceutically acceptable salts, prodrugs and solvates of the foregoing compounds.

In one preferred embodiment of the present invention, the compound of formula 2

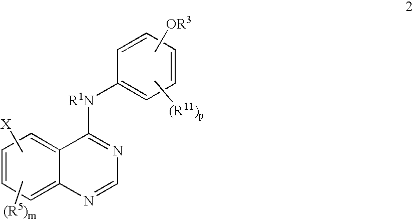

is prepared by reacting a compound of formula 2A

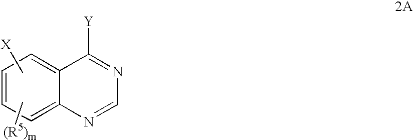

wherein Y is a halide and X, R⁵ and m are as defined for formula 1, with a compound of formula E

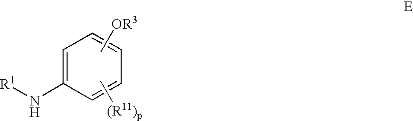

wherein R¹, R³, R¹¹, and p are as defined for formula 1.

In one preferred embodiment the processes of present invention used to make compounds of formula 1, wherein X is Br or I, R⁴ is —C═C—(CR¹⁶R¹⁷)ₜ—R⁹, or —C═C—(CR¹⁶R¹⁷)ₖR¹³ and said reaction is carried out in the presence of a palladium or nickel catalyst selected from the group consisting of Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, BnPdCl(PPh₃)₂, Pd(Otfa)₂, Pd(PPh₃)₂(Otfa)₂, PdCl₂(dppf), Pd(acac)₂, Pd₂(dba)₃CHCl₃, Ni(PPh₃)₄, Pd(dppb),

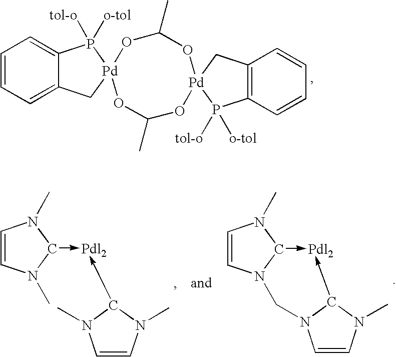

In a preferred embodiment of the processes of the present invention the palladium catalyst is selected from the group consisting of Pd(OAc)₂, Pd₂(dba)₃, and Pd(PPh₃)₄.

In a more preferred embodiment of the processes of the presnt invention the palladium catalyst is selected from the group consisting of Pd(OAc)₂ and Pd(PPh₃)₄.

In a preferred embodiment of the processes of the present invention the ligand is selected from the group consisting a polymer bound phosphine,

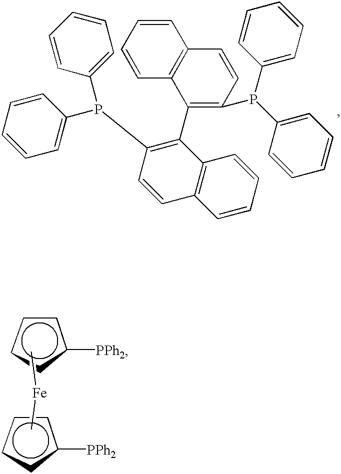

2-methyl-2′-(dicyclohexylphosphino)biphenyl, 2-dimethylamino-2′-(dicyclohexylphosphino)biphenyl, and P(R²²)₃ wherein each R²² is independently selected from the group consisting of 2-methyl-2′-(dicyclohexylphosphino)biphenyl, 2-dimethylamino-2′-(dicyclohexylphosphino)biphenyl, phenyl, o-toluyl, OMe, and furyl,

In a more preferred embodiment of the processes of the present invention the ligand is selected from the group consisting of PPh₃, P(o-Tol)₃, P(o-OMePh)₃, P(2-Furyl)₃,

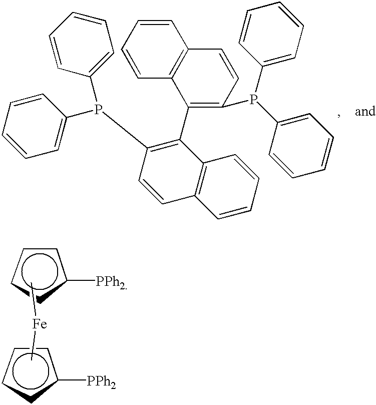

In a most preferred embodiment of the processes of the present invention the ligand is selected from the group consisting of PPh₃, P(o-Tol)₃, and P(2-Furyl)₃.

In a preferred embodiment of the processes of the present invention M is selected from the group consisting of H, Al(R²⁰)₂, Sn(R²¹)₃, MgW, and ZnW and wherein said base is selected from the group consisting of (R)₃N, (R)₂NH, RNH₂, QX, Q₂CO₃, Q₃PO₄, QO₂CR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In a preferrerd embodiment of the processes of the present invention the base is selected from the group consisting of R₄NF, R₄NCl, R₄NBr, Et₃N, Me₂NEt, iPr2NEt, CuBr, CuI, CdCl, CsF, K₂CO₃, Na₃PO₄, Na₂HPO₄, NaOAc, DABCO, and 1,8-(dimethylamino)napthalene, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In a preferred embodiment of the processes of the present invention the base is selected from the group consisting of NaOEt, NaOMe, NaOH, KOH, LiOH, Ca(OH)₂, TIOH, Ba(OH)₂, Et₃N, Me₂NEt, iPr₂NEt, CuBr, CuI, CdCl, CsF, KF, KCl, K₂CO₃, Na₃PO₄, Na₂HPO₄, NaOAc, DABCO, 1,8-(dimethylamino)napthalene, R₄NF, R₄NCl, and R₄NBr wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In a more preferred embodiment of the processes of the present invention the base is selected from the group consisting of Et₃N, Me₂NEt, iPr₂NEt, CuBr, CuI, CdCl, CsF, R₄NF, R₄NCl, R₄NBr, K₂CO₃, Na₃PO₄, Na₂HPO₄, NaOAc, DABCO, and 1,8-(dimethylamino)napthalene, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In an even more preferred embodiment of the processes of the present invention the base is selected from the group consisting of Et₃N, Me₂NEt, K₂CO₃, Na₃PO₄ and NaOAc.

In a preferred embodiment of the processes of the present invention the reaction is carried out in a solvent selected from the group consisting of toluene, benzene, xylene, dimethylformamide, dimethylacetamide, dioxane, tetrahydrofuran, acetonitrile, N-methylpyrrolidinone, dimethylsulfoxide, dimethoxyethane, CH₂Cl₂, CHCl₃, ClCH₂CH₂Cl, N(C₁-C₆ alkyl)₃, N(benzyl)₃, and mixtures thereof.

In a more preferred embodiment of the processes of the present invention the solvent selected from the group consisting of toluene, dimethylformamide, dimethylacetamide, dioxane, tetrahydrofuran, acetonitrile, N-methylpyrrolidinone, dimethoxyethane, ClCH₂CH₂Cl, N(C₁-C₆ alkyl)₃, N(benzyl)₃ and mixtures thereof.

In an even more preferred embodiment of the processes of the present invention the solvent is selected from tetrahydrofuran, dioxane, dimethoxyethane, dimethylformamide, dimethylacetamide, and mixtures thereof.

In a most preferred embodiment of the processes of the present invention the solvent is tetrahydrofuran, water, or a mixture of tetrahydrofuran and water.

In a preferred embodiment of the processes of the present invention the reaction is carried out at a temperature ranging from about 25° C. to about 175° C.

In a preferred embodiment of the processes of the present invention M is B(R¹⁹)₂ and wherein said base is selected from the group consisting of (R)₃N, (R)₂NH, RNH₂, DABCO, 1,8-(dimethylamino)napthalene, QX, Q₂CO₃, Q₃PO₄, Q₂HPO₄, QO₂CR, QOH, and QOR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

In another specific embodiment of the processes of the present invention the compound of formula 1 is prepared by reacting a compound of formula 2 with a compound of formula H—C≡C—(CR¹⁶R¹⁷)ₖR¹³ wherein —(CR¹⁶R¹⁷)ₖR¹³ is selected from the group consisting of —CH₂NHC(O)R¹⁵, —CH₂NHSO₂R¹⁵, and —CH₂NHCO₂R¹⁵ wherein said HC≡CCH₂NHC(O)R¹⁵, HC≡CCH₂NHSO₂R¹⁵, and HC≡CCH₂NHCO₂R¹⁵ are prepared by reacting HC≡CCH₂NH₂ with a compound of formula ClC(O)R¹⁵, ClSO₂R¹⁵, or ClCO₂R¹⁵, respectively.

In another specific embodiment of the processes of the present invention the compound of formula 1 is prepared by reacting a compound of formula 2 with a compound of formula M-C═C—(CR¹⁶R¹⁷)ₖR¹³ wherein —(CR¹⁶R¹⁷)ₖR¹³ is selected from the group consisting of —CH₂NHC(O)R¹⁵, —CH₂NHSO₂R¹⁵, and —CH₂NHCO₂R¹⁵ wherein said H₂C═CCH₂NHC(O)R¹⁵, H₂C═CCH₂NHSO₂R¹⁵ and H₂C═CCH₂NHCO₂R¹⁵ are prepared by reacting HC═CCH₂NH₂ with a compound of formula ClC(O)R¹⁵, ClSO₂R¹⁵, or ClCO₂R¹⁵, respectively.

In one preferred embodiment of the present invention the compound of formula 3

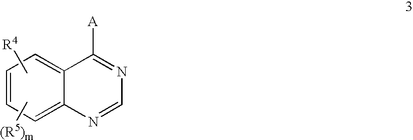

is prepared by reacting a compound of formula

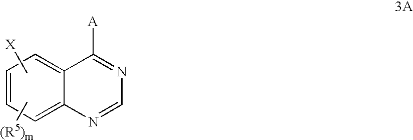

wherein X is a halide, A, R⁵, and m are as defined in claim 1 with a compound of formula MR⁴, wherein R⁴ and M are as defined in claim 1.

In one embodiment of the processes of the present invention the compound of formula 1 is prepared, wherein X is Cl, wherein R⁴ is —C═C—(CR¹⁶R¹⁷)ₜ—R⁹, or —C═C—(CR¹⁶R¹⁷)ₖR¹³, wherein M is B(R¹⁹)₂, and said reaction is carried out in the presence of a catalyst, ligand, base, and solvent, wherein catalyst, ligand, base, and solvent is selected from one of the following groups:

(i) said catalyst is Pd₂(dba)₃ or Pd(OAc)₂, said ligand is 2-methyl-2′-(dicyclohexylphosphino)biphenyl, 2-dimethylamino-2′-(dicyclohexylphosphino)biphenyl, and P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of (R)₄N, M₂CO₃, M₃PO₄, and MX, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1, wherein M is selected from the group consisting of Na, K, Cs and X is halide, and said solvent is selected from the group consisting of toluene, benzene, xylene, DME, acetone, dioxane, DMF, DMAC, NMP, and ACN; or

(ii) said catalyst is selected from the group consisting of Pd(OAc)₂, PdCl₂, Pd(MeCN)₂Cl₂, Pd(PhCN)₂Cl₂, and PdCl₂(PPh₃)₂, said ligand is Ph₄PX, wherein X is selected from the group consisting of Cl, Br, and I, said base is NaOAc or NN dimethylglycine, and said solvent is selected from the group consisting of DMF, DMAC, water, dioxane, THF, ACN, and NMP; or

(iii) said catalyst is selected from the group consisting of Pd(OAc)₂, PdCl₂, Pd(MeCN)₂Cl₂, and Pd(PhCN)₂Cl₂, said ligand is P(OR)₃, wherein R is selected from the group consisting of Et, iPr, Ph, 2,4-dit-BuPh, and Ar, said base is selected from the group consisting of (R)₄N, M₂CO₃ and MO₂CR, wherein M is selected from the group consisting of Na, K, and Cs, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1, and said solvent is selected from the group consisting of DMF, DMAC, water, dioxane, THF, ACN, and NMP; or

(iv) said catalyst is selected from the group consisting of

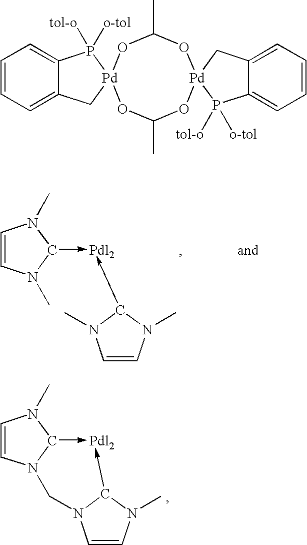

wherein said ligand is P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is (R)₄N or M₂CO₃, wherein M is selected from the group consisting of Na, K, and Cs, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, DME, acetone, Dioxane, DMF, DMAC, and NMP; and

(v) said catalyst is Pd₂(dba)₃, said ligand is Ligand 4 or 5, said base is (R)₄N or M₂CO₃, wherein M is selected from the group consisting of Na, K, and Cs, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1, and said solvent is selected from the group consisting of toluene, benzene, xylene, DME, acetone, Dioxane, DMF, DMAC, and NMP.

In one embodiment of the processes of the present invention the compound of formula 1 is prepared, wherein X is chlorine, R⁴ is —C═C—(CR¹⁶R¹⁷)ₜ—R⁹, or —C═C—(CR¹⁶R¹⁷)ₖR¹³ and M is H and said reaction is carried out in the presence of a catalyst, ligand, base, and solvent mixture comprised of one of the following:

(i) said catalyst is Pd₂(dba)₃ or Pd(OAc)₂, said ligand is 2-methyl-2′-(dicyclohexylphosphino)biphenyl, 2-dimethylamino-2′-(dicyclohexylphosphino)biphenyl, and P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of M₂CO₃, M₃PO₄, and MX wherein M is selected from the group consisting of Na, K, Cs, and (R)₄N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, DME, acetone, Dioxane, DMF, DMAC, NMP, and ACN; or

(ii) said catalyst is selected from the group consisting of Pd(OAc)₂, PdCl₂, Pd(MeCN)₂Cl₂, Pd(PhCN)₂Cl₂, and PdCl₂(PPh₃)₂, said ligand is Ph₄PX, wherein X is selected from the group consisting of Cl, Br, and I, said base is NaOAc or NN dimethylglycine, and said solvent is selected from the group consisting DMF, DMAC, water, dioxane, THF, ACN, and NMP; or

(iii) said catalyst is selected from the group consisting of

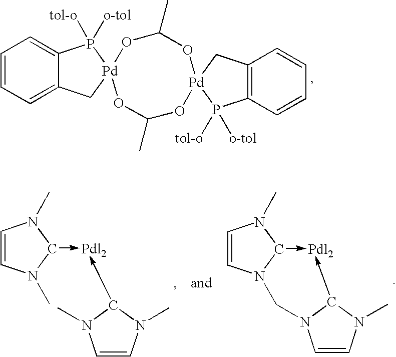

said base is NaOAc, BU₄NBr, hydrazine, or NaOCHO, and said solvent is selected from the group consisting toluene, benzene, xylene, DME, acetone, Dioxane, DMF, DMAC, and NMP; and

(iv) said catalyst is Pd₂(dba)₃, said ligand is

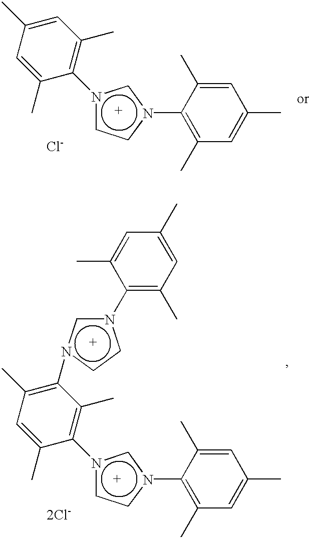

selected from the group consisting NaOAc, Bu₄NBr, hydrazine, and NaOCHO and said solvent is selected from the group consisting toluene, benzene, xylene, DME, acetone, dioxane, DMF, DMAC, and NMP.

In one embodiment of the processes of the present invention the compound of formula 1 is prepared, wherein X is chlorine, R⁴ is —C═C—(CR¹⁶ R¹⁷)ₜ—R⁹, or —C═C—(CR¹⁶R¹⁷)ₖR¹³ and M is Sn(R)₃, said reaction is carried out in the presence of a catalyst, ligand, base, and solvent mixture, wherein said catalyst is Pd₂(dba)₃ or Pd(OAc)₂, said ligand is P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of M₂CO₃, M₃PO₄, MOH and MX, wherein M is selected from the group consisting of Na, K, Cs, and (R)₄N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of DME DMF, DMAC, water, dioxane, THF, ACN, and NMP.

In one embodiment of the processes of the present invention the compound fo formula 1 is prepared, wherein X is Br or I, R⁴ is —C≡C—(CR¹⁶R¹⁷)ₜR⁹ or —C≡C—(CR¹⁶R¹⁷)ₖR¹³, said reaction is carried out in the presence of a catalyst, ligand, base, and solvent mixture, wherein said catalyst is selected from the group consisting of Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, Pd(Otfa)₂, Pd(PPh₃)₂(Otfa)₂, PdCl₂(dppf), Pd(acac)₂, Pd₂(dba)₃-CHCl₃, and Pd(dppb), said ligand is selected from the group consisting of PPh₃, P(o-Tol)₃, P(o-OMePh)₃, P(2-Furyl)₃, said base is selected from the group consisting of (R)₂NH, RNH₂, and (R)₃N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, dimethylformamide, dimethylacetamide, dioxane, tetrahydrofuran, acetonitrile, N-methylpyrrolidinone, dimethoxyethane, acetone, CH₂Cl₂, CHCl₃, and ClCH₂CH₂Cl.

The present invention also relates to a compound of the formula H—C≡C—(CR¹⁶R¹⁷)ₜR⁹, wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

wherein R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups, wherein each R¹ and R² is independently selected from H and C₁-C₆ alkyl, wherein each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5; each R⁶ and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy; or R⁶ and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, and R⁷ are attached, selected from N, N(R¹), O, and S, provided two O atoms, two S atoms or an O and S atom are not attached directly to each other; and t is an integer from 0 to 5.

The present invention also relates to compounds of the formula M-C═C—(CR¹⁶R¹⁷)ₜ—R⁹, wherein M is selected from the group consisting of H, B(R¹⁹)₂, Al(R²⁰)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo wherein R²⁰ is selected from the group consisting of C₁-C₁₀ alkyl, C₁-C₁₀ alkoxy, C₃-C₁₀ cycloalkyl, and halo, wherein R²¹ is C₁-C₁₀ alkyl and wherein W is Cl, Br or I;

wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

wherein R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼwherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups, wherein each R¹ and R² is independently selected from H and C₁-C₆ alkyl, wherein each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR6OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5; each R⁶, and R⁷ is independently selected from H, C₁-C₆ alkyl, 13 (CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy; or R⁶ and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, and R⁷ are attached, selected from N, N(R¹), O, and S, provided two O atoms, two S atoms or an O and S atom are not attached directly to each other; and t is an integer from 0 to 5.

The present invention also relates to compounds of the formula H—C≡C—(CR¹⁶R¹⁷)ₖR¹³, wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

wherein R¹³ is —NR¹R¹⁴ or —OR¹⁴, wherein R¹⁴ is H, R¹⁵, —C(O)R¹⁵, —SO₂R¹⁵, —C(O)NR¹⁵R⁷, —SO₂NR¹⁵R⁷, or —CO₂R¹⁵, wherein R¹⁵ is R¹⁸, —(CR¹R²)ₜ(C₆-C₁₀ aryl), —(CR¹R²)ₜ(4 to 10 membered heterocyclic), wherein each R¹ and R² is independently selected from H and C₁-C₆ alkyl, wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents, wherein R¹⁸ is C₁-C₆ alkyl wherein each carbon not bound to a N or O atom, or to S(O)ⱼ, wherein j is an integer from 0 to 2, is optionally substituted with R¹², wherein R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2, each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;

or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;

each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), and wherein t is an integer from 0 to 5.

The present invention also relates to compounds of the formula M-C═C—(CR¹⁶R¹⁷)ₖR¹³, wherein M is selected from the group consisting of H, B(R¹⁹)₂, Al(R²)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo wherein R²⁰ is selected from the group consisting of C₁-C₁₀ alkyl, C₁-C₁₀ alkoxy, C₃-C₁₀ cycloalkyl, and halo, and wherein R²¹ is C₁-C₁₀ alkyl and wherein W is Cl, Br or I;

wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

wherein R¹³ is —NR¹R¹⁴ or —OR¹⁴, wherein R¹⁴ is H, R¹⁵, —C(O)R¹⁵, —SO₂R¹⁵, —C(O)NR¹⁵R⁷, —SO₂NR¹⁵R⁷, or —CO₂R¹⁵, wherein R¹⁵ is R¹⁸, —(CR¹R²)ₜ(C₆-C₁₀ aryl), —(CR¹R²)ₜ(4 to 10 membered heterocyclic), wherein each R¹ and R² is independently selected from H and C₁-C₆ alkyl, wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents, wherein R¹⁸ is C₁-C₆ alkyl wherein each carbon not bound to a N or O atom, or to S(O)ⱼ, wherein j is an integer from 0 to 2, is optionally substituted with R¹², wherein R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2, each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;

or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;

each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR6 —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), and wherein t is an integer from 0 to 5.

In one preferred embodiment the Suzuki reaction is employed to prepared the compounds of formula 1 by reacting a compound of formula 2 with a compound of formula M-C═C—(CR¹⁶R¹⁷)ₜ—R⁹ or M-C═C—(CR¹⁶R¹⁷)ₖR¹³, wherein M is B. The following review articles, hereby incorporated by reference, identify Pd catalysis and reagents that may be employed in the Suzuki reaction to prepared the compounds of the present invention: (a) Suzuki, A. in Metal-catalyzed Cross-coupling Reactions, Deiderich, F., Stang, P. J., Eds. Wiley, New York, 1998, Chapter 2; and (b) Miyamura, N.; Suzuki, A. Chem. Rev. 1995, 95, 2457-2483. The following articles are hereby incorporated by reference for Suzuki reactions using aryl chlorides: (a) Littke, A. F., Fu, G. C., Angew. Chem. Int. Ed. Engl. 1998, 37, 3387-3388, (b) Wolfed, J. P., Buchwald, S. L., Angew. Chem. Int. Ed. Engl. 1999, 38, 2413-2416, and (c) Littke, A. F., Dai, C., Fu, G. C., J. Am. Chem. Soc., 2000, 122, 4020-4028.

The following table lists preferred combinations of Pd catalysts, ligands, bases and solvents used to prepare the compounds of the present invention using the Suzuki reaction.

| Pd source | Ligand | Base | Solvent |
|---|---|---|---|
| Pd(PPh₃)₄ | PAr₃, preferably PPh₃, | Q(OR)ₙ, wherein n is 1- | Toluene, benzene, or |
| PdCl₂(PPh₃)₂ | P(o-Tol)₃, or P(o- | 3, preferably NaOEt. | xylene, DMF, DMAC, |
| Pd(OAc)₂ | OMePh)₃. | Q(OH)ₘ, preferably | water, dioxane, THF, |
| Pd (Pd/C, Pd black) | P(R)(Ar)₂ | NaOH, KOH, LiOH, | ACN, NMP, MeOH, |
| PdCl₂(dppf) | P(R)₂(Ar) | CaOH₂, TIOH, Ba(OH)₂ | EtOH, iPrOH, DME, or |
| | P(R)₃ | (R)₃N preferably Et₃N or | acetone. |
| | Dppf, dppe, dppb, or | Me₂NEt. | |
| | dppp. | QF or QCl | |
| | Polymer bound | Q(CO₃) | |
| | phosphines | QH(PO₄) | |
| | | Q(OCOR) for example | |
| | | NaOAc. | |

The following boranes may be employed to provide the vinyl borane coupling partner in the Suzuki reaction from the corresponding acetylene: catecholborane, 9-borabicyclo[3.3.1]nonane [9-BBN], dithexylborane, diisoamylborane, dicyclohexylborane, or HB(OR)₂ wherein R is C₁-C₁₀ alkyl, phenyl, and benzyl or H.

In one preferred embodiment when X is Cl the following Table lists the Pd catalyst, ligand, based and solvent which may be employed for the preparation of the compounds of formula 1. Applicants' also incorporated by reference the related information disclosed in Buchwald, S. L.; Fox, J. M. The Strem Chemiker, Vol 18, no. 1, p. 1-14, 2000.

| Pd source | Ligand | Base | Solvent |
|---|---|---|---|
| Pd₂(dba)₃ or Pd(OAc)₂ | P(R)₃, preferably | Q(CO₃) preferably | Toluene, benzene, |
| | P(t-Bu)₃ or P(i-Pr)₃. | Cs₂(CO₃). | xylene, DME, acetone |
| | | | Dioxane, DMF, |
| | | | DMAC, NMP, or ACN. |
| Pd₂(dba)₃ or Pd(OAc)₂ | 2-methyl-2′-(dicyclo- | Q(CO₃), preferably | Toluene, benzene, |
| | hexylphosphino)biphe | Cs₂(CO₃), QF, | xylene, DME, acetone |
| | nyl or 2- | preferably CsF, and | Dioxane, DMF, |
| | dimethylamino-2′- | QPO₄, preferably | DMAC, NMP, or CAN. |
| | (dicyclohexyl- | K₃PO₄. | |
| | phosphino)biphenyl | | |
| Pd(Oac)₂, PdCl₂, | Ph₄PX, wherein X is | NaOAc or | DMF, DMAC, water, |
| Pd(MeCN)₂Cl₂, | Cl, Br, or I. | N,N-dimethylglycine | dioxane, THF, ACN, |
| Pd(PhCN)₂Cl₂, or | | | or NMP. |
| PdCl₂(PPh₃)₂ | | | |
| Pd(OAc)₂, PdCl₂, | P(OR)₃, wherein R is | Q(OCOR), perferably | DMF, DMAC, water, |
| Pd(MeCN)₂Cl₂, or | Et, iPr, Ph, 2,4-dif- | NaOAc, and Q(CO₃), | dioxane, THF, ACN, |
| Pd(PhCN)₂Cl₂. | BuPh, Ar, or Dippb. | preferably Na(CO₃). | or NMP. |
| Palladacycle 1 or | No Ligand | QCO₃, preferably | Toluene, benzene, |
| Catalysts 1 or 2. | | Cs₂CO₃ and K₂CO₃. | xylene, DME, acetone |
| | | | Dioxane, DMF, |
| | | | DMAC, or NMP. |
| Pd₂(dba)₃ | Ligand 4 or 5 | No Base | Toluene, benzene, |
| | | | xylene, DME, acetone |
| | | | Dioxane, DMF, |
| | | | DMAC, or NMP. |

In one preferred embodiment the Heck reaction is employed to prepared the compounds of formula 1 by reacting a compound of formula 2 with a compound of formula M-C═C—(CR¹⁶R¹⁷)ₜ—R⁹ or M-C═C—(CR¹⁶R¹⁷)ₖR¹³ wherein M is H is employed to prepared the compound of formula 1. The following review articles, hereby incorporated by reference, identify reagents that may be employed in the Heck reaction to prepared the compounds of the present invention: (a) Heck, R. F. in Comprehensive Organic Synthesis; Trost, B. M., Ed.; Pergamon: New York, 1991; Vol. 4, Chapter 4.3; (b) Bräse, S.; deMeijere, A. in Metal-catalyzed Cross-coupling Reactions; Deiderich, F.; Stang, P. J., Eds.; Wiley: New York, 1998, Chapter 3; (c) Cabri, W.; Candiani, I. Acc. Chem. Res. 1995, 28, 2-7; and (d) deMeijere, A.; Meyer, F. E. Angew. Chem. Int. Ed. Engl. 1994, 33, 2379-2411.

In one preferred embodiment of the process of the present invention the Heck reactions employ aryl chlorides. The following articles disclose the use of aryl chlorides in the Heck reaction, which are hereby incorporated by reference: (a) Riermeier, T. H.; Zapf, A.; Beller, M. Top. Catal. 1997, 4, 301-309; (b) Littke, A. F.; Fu, G. C. J. Org. Chem. 1999, 64, 10-11; (c) Reetz, M. T.; Lohmer, G.; Schwickardi, R. Angew. Chem. Int. Ed. 1998, 37, 481-483; (d) Beller, M.; Zapf, A. Synlett 1998, 792-793; (e) Ben-David, Y.; Portnoy, M.; Gozin, M.; Milstein, D. Organometallics 1992, 11, 1995-1996; (f) Portnoy, M.; Ben-David, Y.; Milstein, D. Organometallics 1993, 12, 4734-4735; (g) Portnoy, M.; Ben-David, Y.; Rousso, I.; Milstein, D. Organometallics 1994, 13, 3465-3479; (h) Herrmann, W. A.; Brossmer, C.; Öfele, K.; Reisinger, C. -P.; Priermeier, T.; Beller, M.; Fischer H. Angew. Chem. Int. Ed. Engl. 1995, 34, 1844-1848; (i) Herrmann, W. A.; Elison, M.; Fischer J.; Köcher, C.; Artus, G. R. J. Angew. Chem. Int. Ed. Engl. 1995, 34, 2371-2374; and (j) Herrmann, W. A.; Brossmer, C.; Reisinger, C. -P.; Riermeier, T. H.; Öfele, K.; Beller, M. Chem. Eur. J. 1997, 3, 1357-1364.

The following table lists preferred Pd catalysts, ligands, bases, and solvents from Bräse, S.; deMeijere, A. in Metal-catalyzed Cross-coupling Reactions; Deiderich, F.; Stang, P. J., Eds.; Wiley: New York, 1998; Chapter 3, pages 108-109 for use in the Heck reaction.

| Pd source | Ligand | Base | Solvent |
|---|---|---|---|
| Pd(PPh₃)₄ | PAr₃, preferably PPh₃, | DABCO, proton | Toluene, benzene, |
| PdCl₂(PPh₃)₂, or | P(o-Tol)₃, P(o- | sponge, (R)₂NH, | xylene, DMF, DMAC, |
| BnPdCl(PPh₃)₂. | OMePh)₃, P(2-Furyl)₃, | (R)NH₂ | water, dioxane, THF, |
| Pd(OAc)₂, | BINAP, dppf, dppe, | (R)₃N, | ACN, NMP, DMSO, |
| Pd(O₂CCF₃)₂, or | dppb, or dppp. | QX, wherein X is F, | MeOH, EtOH, iPrOH, |
| Pd(PPh₃)₂(O₂CCF₃)₂. | Polymer bound | Cl, or Br, | DME, acetone. |
| Pd (Pd/C, Pd black, | phosphines | Q(CO₃) | CH₂Cl₂, CHCl₃, |
| Pd on other solid | | QH(PO₄) | ClCH₂CH₂Cl, NR₃, |
| supports such as | | Q(OCOR), preferably | preferably NEt₃ or |
| silica, graphite, clay). | | NaOAc. | iPr₂NEt. |
| PdCl₂, Pd(MeCN)₂Cl₂, | | | |
| or Pd(PhCN)₂Cl₂. | | | |
| PdCl₂(dppf), | | | |
| Pd(acac)₂, Pd₂(dba)₃, | | | |
| Pd(dppb), Pd₂(dba)₃- | | | |
| or CHCl₃. | | | |
| P(Ar)₃, preferably | | | |
| PPh₃, P(o-Tol)₃, P(o- | | | |
| OMePh)₃, P(2-Furyl)₃, | | | |

In one preferred embodiment when X is Cl the following Table lists the Pd catalyst, ligand, based and solvent, which may be employed for the preparation of the compounds of formula 1 using the Heck reaction.

| Pd source | Ligand | Base | Solvent |
|---|---|---|---|
| Pd₂(dba)₃ or Pd(OAc)₂ | P(R)₃, preferably P(t- | Q(CO₃), preferably | Toluene, benzene, |
| | Bu)₃ or P(i-Pr)₃. | Cs₂(CO₃). | xylene, DME, acetone |
| | | | Dioxane, DMF, |
| | | | DMAC, NMP, or ACN. |
| Pd(OAc)₂, PdCl₂, | Ph₄PX, wherein X is | NaOAc or | DMF, DMAC, water, |
| Pd(MeCN)₂Cl₂, | Cl, Br, or I. | NN dimethylglycine | dioxane, THF, ACN, |
| Pd(PhCN)₂Cl₂, or | | | or NMP. |
| PdCl₂(PPh₃)₂ | | | |
| Pd(OAc)₂, PdCl₂, | P(OR)₃, wherein R is | Q(OCOR), perferably | DMF, DMAC, water, |
| Pd(MeCN)₂Cl₂, or | Et, iPr, Ph, 2,4-dit- | NaOAc, and Q(CO₃), | dioxane, THF, ACN, |
| Pd(PhCN)₂Cl₂, | BuPh, Ar, | preferably Na₂CO₃. | or NMP. |
| | Or dippb. | | |
| Palladacycle 1 | No Ligand | NaOAc, Bu₄NBr, | DMAc, DMF, or NMP. |
| Catalysts 1-2 | | hydrazine, or | |
| | | NaOCHO. | |
| Pd₂(dba)₃ | Ligand 1 | NaOAc, Bu₄NBr, | DMAc, DMF, or NMP. |
| | | hydrazine, or | |
| | | NaOCHO. | |

In one embodiment of the present invention the Stille Coupling reaction is employed to prepared the compounds of formula 1 by reacting a compound of formula 2 with a compound of formula M-C═C—(CR¹⁶R¹⁷)ₜ—R⁹ or M-C═C—(CR¹⁶R¹⁷)ₖR¹³wherein M is Sn, in the presence of a ligand, base and solvent. The following review, Mitchell, T. N. in Metal-catalyzed Cross-coupling Reactions; Deiderich, F.; Stang, P. J., Eds.; Wiley: New York, 1998, Chapter 4, hereby incorporated by reference, identify reagents that may be employed in the Stille Coupling reaction to prepared the compounds of the present invention.

The following table lists preferred combinations of Pd catalysts, ligands, bases and solvents used to prepare the compounds of the present invention using the Stille Coupling reaction.

| Pd source | Ligand | Base | Solvent |
|---|---|---|---|
| Pd(PPh₃)₄ | P(Ar)₃, preferably | DABCO, proton | Toluene, benzene, |
| PdCl₂(PPh₃)₂, or | P(Ph)₃, P(o-Tol)₃, P(o- | sponge, (R)₂NH, | xylene, DMF, DMAC, |
| BnPdCl(PPh₃)₂. | OMePh)₃, or P(2- | RNH₂, | water, dioxane, THF, |
| Pd(Oac)_{2,} | Furyl)₃, | (R)₃N, preferably Et₃N, | ACN, NMP, DMSO, |
| Pd(O₂CCF₃)₂, or | BINAP, | or Me₂NEt. | MeOH, EtOH, iPrOH, |
| Pd(PPh₃)₂(O₂CCF₃)₂. | As(Ph)₃ | QX, wherein X is F, | DME, acetone. |
| Pd (Pd/C, Pd black, | Dppf, dppe, dppb, or | Cl, or Br. | CH₂Cl₂, CHCl₃, |
| Pd on other solid | dppp. | CuBr, CuI, or CdCl. | ClCH₂CH₂Cl, N(R)₃, |
| supports such as | | Q(CO₃) | preferably NEt₃ or |
| silica, graphite, clay). | | QH(PO₄) | iPr₂NEt. |
| PdCl₂, Pd(MeCN)₂Cl₂, | | Q(OCOR), perferably | |
| Pd(PhCN)₂Cl₂. | | NaOAc. | |
| PdCl₂(dppf), | | | |
| Pd(acac)₂, Pd₂(dba)₃, | | | |
| Pd(dppb), or | | | |
| Pd₂(dba)₃-CHCl₃. | | | |

In one preferred embodiment when X is Cl the following Table lists the Pd catalyst, ligand, based and solvent, which may be employed for the preparation of the compounds of formula 1 using the Stille Coupling reaction.

| Pd source | Ligand | Base | Solvent |
|---|---|---|---|
| Pd₂(dba)₃ or Pd(OAc)₂. | P(R)₃, preferably | QF, preferably CsF, | DME DMF, DMAC, |
| | P(t-Bu)₃ or P(i-Pr)₃. | Q(CO₃), preferably | water, dioxane, THF, |
| | | Cs₂(CO₃), (R)₃N, | ACN, or NMP. |
| | | Or QOH. | |

In one embodiment of the present invention a metal catalyzed cross coupling reaction is employed to prepared compounds of formula 1 by reacting a a compound of formula M-C═C—(CR¹⁶R¹⁷)ₜ—R⁹ or M-C═C—(CR¹⁶R¹⁷)ₖR¹³ wherein M is Mg, Zn, Zr or Al, with a compound of formula 2 in the presence of a ligand, base and solvent. The following table lists preferred combinations of Pd catalysts, ligands, bases and solvents used to prepare the compounds of the present invention using a metal catalyzed cross coupling reaction, wherein M is Mg, Zn, Zr or Al. Applicants also incorporate by reference, Negishi, E-i.; Liu, F. in Metal-catalyzed Cross-coupling Reactions; Deiderich, F.; Stang, P. J., Eds.; Wiley: New York, 1998, Chapter 1.

| Pd source | Ligand | Base | Solvent |
|---|---|---|---|
| Pd(PPh₃)₄ | PAr₃, preferably | None, or | Toluene, benzene, or |
| PdCl₂(PPh₃)₂, or | P(Ph)₃, P(o-Tol)₃, P(o- | QX, wherein X is F, | xylene. |
| BnPdCl(PPh₃)₂. | OMePh)₃, or P(2- | Cl, or Br, CuBr, CuI, | Dioxane or THF. |
| Pd(OAc)₂, | Furyl)₃. | CdCl, or ZnCl₂. | DME |
| Pd(O₂CCF₃)₂, or | BINAP. | | CH₂Cl₂, CHCl₃, or |
| Pd(PPh₃)₂(O₂CCF₃)₂. | As(Ph)₃ | | ClCH₂CH₂Cl. |
| Pd (Pd/C, Pd black, | dppf, dppe, dppb, or | | N(R)₃, preferably NEt₃ |
| Pd on other solid | dppp. | | or iPrNEt. |
| supports such as | Polymer bound | | |
| silica, graphite, clay) | phosphines | | |
| PdCl₂, Pd(MeCN)₂Cl₂, | | | |
| or Pd(PhCN)₂Cl₂. | | | |
| PdCl₂(dppf), | | | |
| Pd(acac)₂, Pd₂(dba)₃, | | | |
| Pd(dppb), or | | | |
| Pd₂(dba)₃-CHCl₃. | | | |
| Ni(PPh₃)₄ | | | |

In one embodiment of the present invention employing the Sonogashira Coupling reaction is employed to prepared compounds of formula 1 by reacting a compound of formula H—C≡C—(CR¹⁶R¹⁷)ₜR⁹ or H—C≡C—(CR¹⁶R¹⁷)ₖR¹³ with a compound of formula 1 in the presence of CuI or CuBr. A ligand, base and solvent are also employed. The following review, Sonogashira, K. in Metal-catalyzed Cross-coupling Reactions; Deiderich, F.; Stang, P. J., Eds.; Wiley: New York, 1998; Chapter 5, hereby incorporated by reference, identify reagents that may be employed in the Sonogashira Coupling reaction to prepared the compounds of the present invention. The following table lists preferred combinations of Pd catalysts, ligands, bases and solvents used to prepare the compounds of the present invention using the Sonogashira Coupling reaction.

| Pd source | Ligand | Base | Solvent |
|---|---|---|---|
| Pd(PPh₃)₄ | P(Ar)₃, preferably | (R)₂NH, preferably, | Toluene, benzene, or |
| PdCl₂(PPh₃)₂ | P(Ph)₃. | Et₂NH or iPr₂NH. | xylene. |
| Pd(OAc)₂, | Polymer bound | Piperidine, or | DMF, DMAC, water, |
| Pd(O₂CCF₃)₂, or | phosphines | pyrrolidine. | dioxane, THF, ACN, |
| Pd(PPh₃)₂(O₂CCF₃)₂. | | RNH₂, preferably | NMP, or DMSO. |
| Pd (Pd/C, Pd black, | | BuNH₂ or iPrNH₂. | MeOH or EtOH. |
| Pd on other solid | | (R)₃N preferably | DME or acetone. |
| supports such as | | (Et)₃N or (Me)₂NEt. | CH₂Cl₂, CHCl₃, or |
| silica, graphite, clay) | | | ClCH₂CH₂Cl. |
| PdCl₂, Pd(MeCN)₂Cl₂, | | | N(R)₃, preferably NEt₃ |
| or Pd(PhCN)₂Cl₂. | | | or iPrNEt. |
| PdCl₂(dppf), | | | |
| Pd(acac)₂, Pd₂(dba)₃, | | | |
| Pd(dppb), or | | | |
| Pd₂(dba)₃-CHCl₃. | | | |

Preferably, the palladium catalyst employed in the present invention is a palladium(0) catalyst, more preferably the palladium(0) catalyst is tretrakis(triphenylphosphine)palladium(0). This may be added to the reaction mixture directly or generated in situ by adding triphenylphosphine and palladium acetate which is converted to palladium(0) species under the reaction conditions.

This compounds of formula 1 may be used to treat abnormal cell growth in a mammal, including a human, comprising administering to said mammal an amount of a compound of the formula 1, as defined above, or a pharmaceutically acceptable salt, solvate or prodrug thereof, that is effective in treating abnormal cell growth. The abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In other embodiments employing the compounds of formula 1 the abnormal cell growth is a benign proliferative disease, including, but not limited to, psoriasis, benign prostatic hypertrophy or restinosis.

The compounds of formula 1 may be used in the treatment of a disorder associated with angiogenesis in a mammal, including a human, comprising administering to said mammal an amount of a compound of the formula 1, as defined above, or a pharmaceutically acceptable salt, solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “halo”, as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl” or “Ar”, as used herein, unless otherwise indicated, includes an organic radical derived from an aromatic hydrocarbon by removal of one hydrogen, such as phenyl or naphthyl. “Aryl” or “Ar” are optionally substituted with 1 to 4 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-G₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5, wherein t, R¹, R², R⁶, and R⁷ are as defined for formula 1.

The term “alkoxy”, as used herein, unless otherwise indicated, includes —O-alkyl groups wherein alkyl is as defined above.

The term “4 to 10 membered heterocyclic”, as used herein, unless otherwise indicated, includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The term “Me” means methyl, “Et” means ethyl, and “Ac” means acetyl.

The term “DME”, as used herein, unless otherwise indicated, means dimethoxyethane.

The term “DMF”, as used herein, unless otherwise indicated, means dimethylformamide.

The term “DMAC”, as used herein, unless otherwise indicated, means dimethylacetamide.

The term “ACN”, as used herein, unless otherwise indicated, means acetonitrile.

The term “NMP”, as used herein, unless otherwise indicated, means N-methylpyrrolidinone.

The term “DMSO”, as used herein, unless otherwise indicated, means dimethylsulfoxide.

The term “BINAP”, as used herein, unless otherwise indicated, is represented by the following formula:

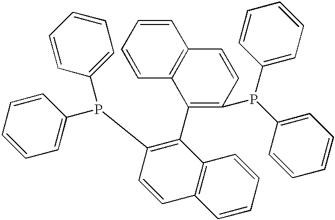

The term “DABCO”, as used herein, unless otherwise indicated, means 1,4-diazabicyclo[2.2.2]octane.

The term “DBA”, as used herein, unless otherwise indicated, means dibenzanthracene.

The term “dppe”, as used herein, unless otherwise indicated, means Ph₂P(CH₂)₂PPh₂.

The term “dppp”, as used herein, unless otherwise indicated, means Ph₂P(CH₂)₃PPh₂.

The term “dppb”, as used herein, unless otherwise indicated, means Ph₂P(CH₂)₄PPh₂.

The term “dippb”, as used herein, unless otherwise indicated, means iPr₂P(CH₂)₄PiPr₂.

The term “dppf”, as used herein, unless otherwise indicated, is represented by the following formula:

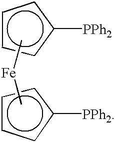

The term “paladacycle 1”, as used herein, unless otherwise indicated, is represented by the following formula:

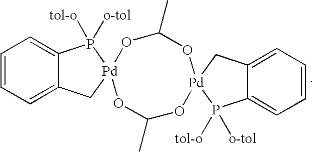

The term “protein sponge”, as used herein, unless otherwise indicated, means 1,8-bis(dimethylamino)naphthalene.

The term “catalyst 1”, as used herein, unless otherwise indicated, is represented by the following formula:

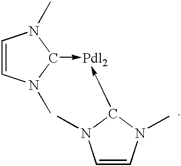

The term “catalyst 2”, as used herein, unless otherwise indicated, is represented by the following formula

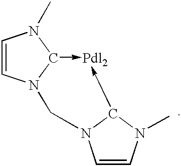

The term “ligand 1”, as used herein, unless otherwise indicated, is represented by the following formula

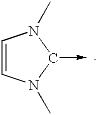

The term “ligand 4”, as used herein, unless otherwise indicated, is represented by the following formula

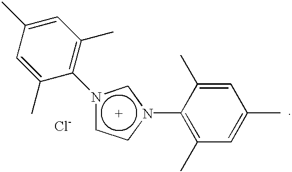

The term “ligand 5”, as used herein, unless otherwise indicated, is represented by the following formula

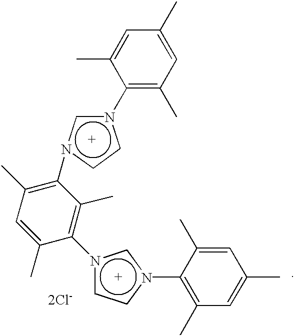

The term “Otfa”, as used herein, unless otherwise indicated, means O₂CCF₃.

The term “R”, as used herein, unless otherwise indicated, means it is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, wherein R¹ and R² are as defined above for formula 1.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)]salts. The compounds of the present invention that include a basic moiety, such as an amino group, may form pharmaceutically acceptable salts with various amino acids, in addition to the acids mentioned above.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

The subject invention also includes isotopically-labelled compounds, and the pharmaceutically acceptable salts, solvates and prodrugs thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

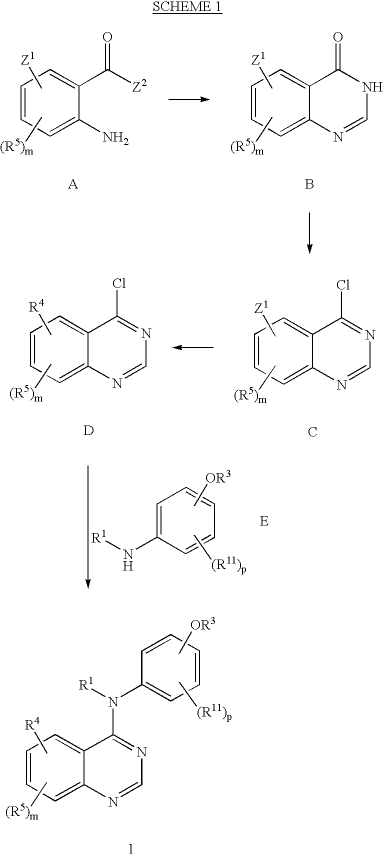

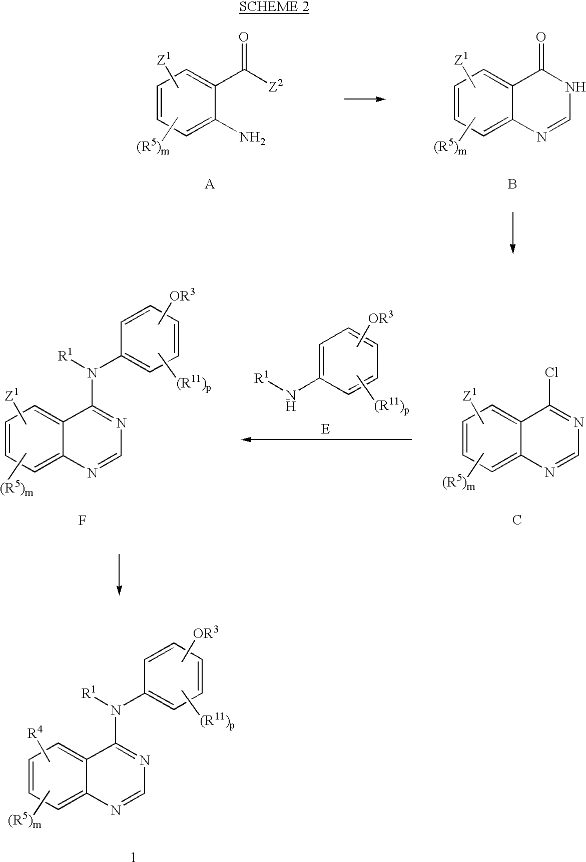

### DETAILED DESCRIPTION OF THE INVENTION

General synthetic methods which may be referred to for preparing the compounds of the present invention are provided in U.S. Pat. No. 5,747,498 (issued May 5, 1998), U.S. patent application Ser. No. 08/953078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in U.S. patent application Ser. Nos. 09/488,350 (filed Jan. 20, 2000) and 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem. 1986, 51, 5, p. 616. Palladium-catalyzed boronic acid couplings are described in Miyaura, N., Yanagi, T., Suzuki, A. Syn. Comm. 1981, 11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

Alternatively terminal alkynes may be prepared by a two step procedure. First, the addition of the lithium anion of TMS (trimethylsilyl) acetylene to an appropriately substituted/protected aldehyde as in: Nakatani, K. et. al. Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al. Tet. Lett., 31, 1, 1990, 59.

Starting materials, the synthesis of which is not specifically described above, are either commercially available or can be prepared using methods well known to those of skill in the art.

In each of the reactions discussed or illustrated in the Schemes above, pressure is not critical unless otherwise indicated. Pressures from about 0.5 atmospheres to about 5 atmospheres are generally acceptable, and ambient pressure, i.e., about 1 atmosphere, is preferred as a matter of convenience.

With reference to Scheme 1 above, the compound of formula 1 may be prepared by coupling the compound of formula D wherein R⁴ and R⁵ are defined above, with an amine of formula E wherein R¹, R³ and R¹¹ are as defined above, in an anhydrous solvent, in particular a solvent selected from DMF (N,N-dimethylformamide), DME (ethylene glycol dimethyl ether), DCE (dichloroethane) and t-butanol, and phenol, or a mixture of the foregoing solvents, a temperature within the range of about 50-150° C. for a period ranging from 1 hour to 48 hours. The heteroaryloxyanilines of formula E may be prepared by methods known to those skilled in the art, such as, reduction of the corresponding nitro intermediates. Reduction of aromatic nitro groups may be performed by methods outlined in Brown, R. K., Nelson, N. A. J. Org. Chem. 1954, p. 5149; Yuste, R., Saldana, M, Walls, F., Tet. Lett. 1982, 23, 2, p. 147; or in WO 96/09294, referred to above. Appropriate heteroaryloxy nitrobenzene derivatives may be prepared from halo nitrobenzene precursors by nucleophilic displacement of the halide with an appropriate alcohol as described in Dinsmore, C. J. et. al., Bioorg. Med. Chem. Lett., 7, 10, 1997, 1345; Loupy, A. et. al., Synth. Commun., 20, 18, 1990, 2855; or Brunelle, D. J., Tet. Lett., 25, 32, 1984, 3383. Compounds of formula E in which R¹ is a C₁-C₆ alkyl group may be prepared by reductive amination of the parent aniline with R¹CH(O). The compound of formula D may be prepared by treating a compound of formula C, wherein Z¹ is an activating group, such as bromo, iodo, —N₂, or —OTf (which is —OSO₂CF₃), or the precursor of an activating group such as NO₂, NH₂ or OH, with a coupling partner, such as a terminal alkyne, terminal alkene, vinyl halide, vinyl stannane, vinylborane, alkyl borane, or an alkyl or alkenyl zinc reagent. The compound of formula C can be prepared by treating a compound of formula B with a chlorinating reagent such as POCl₃, SOCl₂ or ClC(O)C(O)Cl/DMF in a halogenated solvent at a temperature ranging from about 60° C to 150° C for a period ranging from about 2 to 24 hours. Compounds of formula B may be prepared from a compound of formula A wherein Z¹ is as described above and Z² is NH₂, C₁-C₆ alkoxy or OH, according to one or more procedures described in WO 95/19774, referred to above.

The compounds and reactions in Scheme 2 may be prepared using the methods described for Scheme 1, with one change to the reaction scheme. The compound of formula C is treated with the heteroaryloxyanilines of formula E to form the compound formula F prior to the reaction of the Z¹ activating group with a coupling partner as described above in Scheme 1.

Any compound of formula 1 can be converted into another compound of formula 1 by standard manipulations to the R⁴ group. These methods are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively; c) treatment of phenyl (or substituted phenyl) carbamates with primary of secondary amines to form the corresponding ureas as in Thavonekham, B et. al. Synthesis (1997), 10, p1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formulas 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product. Since a single compound of the present invention may include more than one acidic or basic moieties, the compounds of the present invention may include mono, di or tri-salts in a single compound.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

The present invention is illustrated by the following Examples. It will be understood, however, that the invention is not limited by the specific details of the following Examples.

### EXAMPLE 1

### 6-Iodo-[3-methyl-4-(6-methyl-pyridine-3-yloxy)-phenylamino]-quinazoline

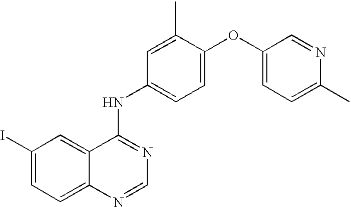

A 3 neck round bottom flask was fitted with a mechanical stirrer and kept under N₂. The flask was charged with the chloroquinazoline (10.0 g, 34.43 mol) and dry THF (35 ml). The 3-amino-4-methylpyridine (7.38 g, 34.43 mmol) and dry THF (45 ml) were added and the yellow suspension was heated to reflux. After 15 min most of the reactants went into solution and a fine yellow suspension was obtained. After 25 min, the internal temperature of the reaction mixture was 56° C., and precipitation of the desired product started. Heating was continued for a further 2 hours and the reaction mixture was allowed to cool to room temperature while remaining in the oil bath. Yellow crystals were collected by filtration, washed with cold (0° C.) THF (1×10 ml) and dried at 50° C., p<200 mbar. The title compound was obtained as light yellow crystals (15.75 g, 98%). Rf=0.45 (EtOAc/MeOH=9/1). ¹H NMR (CDCl₃, 300 MHz): δ=11.40 (br, s, 1H, NH), 9.29 (d, J=Hz, 1H, H-2), 8.91 (s, 1H, H-2″), 8.36-8.32 (m, 2H, H-7, H-8), 7.74-7.73 (m, 2H, H-4″, H-5), 7.62 (dd, J₁=8.7 Hz, J₂=2.6 Hz, 1H, H-5″) 7.49-7.46 (m, 2H, H-6′, H-5), 7.06 (d, J=8.7 Hz, 1H, H-2′), 2.54 (s, 3H, CH₃), 2.26 (s, 3H, CH₃). ¹³C NMR (CDCl₃+D₆-DMSO, 75 MHz): δ=159.51, 153.63, 153.17, 152.82, 152.70, 145.26, 141.37, 138.01, 134.75, 134.65, 131.05, 129.10, 128.74, 126.77, 124.86, 124.43, 120.41, 116.98, 94.89, 23.54, 17.67.

The title compound had a t_{R} (min) of 12.13 under the following RP-HPLC conditions: Symmetry Shield RP18, 75×4.6 mm; Flow 1.0 mL/min; 205/210/220/245 nm; Temp. 25° C.; Injection Volume: 10 μL of a ca. 0.5% solution in ACN/H₂O 9/1; Eluent: B: ACN, C: 0.01 mmol NH₄OAc in H₂O pH=6.0; and Gradient: 0 min: B=30%, C=70%; and 20 min: B=85%, C=15%.

### EXAMPLE 2

### 2-Methoxy-acetic Acid Propargylamide

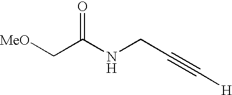

A solution of methoxy acetyl chloride (12.5 ml, 0.137 mol, 1.2 equiv.) in dry CH₂Cl₂ (45 ml) kept under N₂ was cooled to −40° C. A solution of propargylamine (7.98 ml, 0.125 mol. 1.0 equiv.) in dry CH₂Cl₂ (40 ml) was added over 45 minutes keeping the temperature less than −25° C. After 15 minutes triethylamine (17.4 ml, 0.125 mol, 1.0 equiv.) was added over 45 minutes keeping the temperature less than −25° C. The reaction mixture was warmed to room temperature. TLC after 3 hours showed conversion complete. The reaction mixture was quenched with H₂O (50 ml) and the organic phase was washed with half-saturated NaCl solution, filtered through cotton wool and concentrated at a temperature of 40° C. and pressure of greater than 650 mbar. The crude compound was purified by short path distillation (boiling point of 49° C. and p of 0.09 mbar). The title compound was obtained as a colorless liquid (7.84 g, 50%) which crystallized upon standing.

R_{f}=0.36 (heptane/EtOAc=7/3).

¹H NMR (CDCl₃, 300 MHz): δ=6.72 (br, s, 1H, N—H), 4.09 (dd, J₁=5.5 Hz, J₂=2.6 Hz, 2H, CH₂—NH), 3.92 (s, 2H, CH₂—OMe), 3.43 (s, 3H, OCH₃), 2.24 (t, J=2.6 Hz, 1H, alkyne CH). ¹³C-NMR (CDCl₃, 75 MHz): δ=169.14 (C═O), 79.11 (C-2′), 71.63 (C-2), 71.41 (C-3′), 59.04 (OCH₃), 28.26 (C-1′).

Gas chromatography was used to determine the t_{R} (min) of 6.42 under the conditions shown in the table below.

| | |
|---|---|
| Column | DB-5 (30 m × 0.32 mm, 0.25 μm film thickness) |
| Injector | Split, initial Temp. 250° C. |
| Split ratio | 60.243:1 |
| Split flow | 108.3 ml/min, gas type: hydrogen |
| Oven | 60° C., 1 min, 10° C./min, 290° C., 10 min |
| Inject-Temp | 250° C. |
| Detector (FID) | Detector Temp. 250° C. |
| Detector flow | H₂: 40.0 ml/min, air: 450 ml/min |
| Makeup flow | N₂: 45.0 ml/min |

### EXAMPLE 3

### Preparation of 6-(N-Methoxyacetyl-3-amino-propen-1-yl)-4-[3-methyl-4-(6-methyl-pyridine-3-yloxy)-phenylamino]-quinazoline Using Suzuki Coupling Reaction

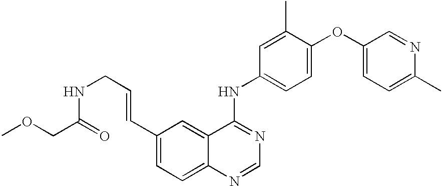

2-methyl-2-butene (0.59 ml, 5.60 mmol, 2.8 equiv.) was added over 1 hour to a cold (0-5° C.) solution of BH₃*THF complex (1.0 M sol, 3.0 ml, 3.0 mmol, 1.5 equiv.) kept under N₂. The reaction mixture was stirred at this temperature for 30 minutes followed by the addition of 2-Methoxy-acetic acid propargylamide (255 mg, 2 mmol, 1.0 equiv.) dissolved in dry THF (1 ml) over 15 minutes. The ice-bath was removed and the reaction mixture was warmed to room temperature over 20 minutes. The reaction mixture was then heated at 35° C. for 1 hour. K₂CO₃ (0.55 g, 4 mmol, 2.0 equiv.) dissolved in degassed H₂O (1.2 ml) was added over 30 minutes to the reaction mixture. During the addition of the first half gas evolution was observed which seized during further addition. 6-Iodo-[3-methyl-4-(6-methyl-pyridine-3-yloxy)-phenylamino]-quinazoline (1.41 g, 3 mmol, 1.5 equiv.) was added in three portions giving a yellow suspension. PPh₃ (21 mg, 0.08 mmol, 4 mol %) and Pd(OAc)₂ (4.5 mg, 0.02 mmol, 1 mol %) were added each in one portion and the reaction mixture was heated to reflux (65-68° C.). After about 30 minutes a yellow solution was obtained and the reaction was monitored by HPLC assay. After 18 hours the reaction mixture was cooled to room temperature followed by the addition of half-saturated NaCl solution (10 ml) and EtOAc (10 ml). The organic phase was separated, washed with H₂O (5 ml) and concentrated at 50° C. and a pressure of less than 200 mbar. Purification by plug filtration, SiO₂, EtOAc/MeOH=9/1. The title compound was obtained as light yellow crystals (0.55 g, 59%). R_{f}=0.16 (EtOAc/MeOH=9/1). ¹H-NMR (CDCl₃, 250 MHz): δ=8.71 (s, 1H, H-2), 8.25 (d, J=1.7 Hz, 1H, H-8), 7.90 (s, 1H, H-7), 7.82 (s, 1H, NH), 7.79 (s, 1H, H-5), 7.66 (d, J=2.5 Hz, 1H, H-4″), 7.54 (dd, J₁=8.7 Hz, J₂=2.6 Hz, 1H, H-5″), 7.15-7.07.(m, 2H, H-5′, H-6′), 6.91 (d, J=8.7 Hz, 1H, H-2′), 6.83 (bt, 1H, NH), 6.65 (d, J=15.9 Hz, 1H, H-9), 6.34 and 6.29 (dt, J₁=15.9 Hz, J₂=6.1 Hz, 1H, H-10), 4.14 (dt, J=6.1 Hz, 2H, CH₂OMe), 3.97 (s, 2H, CH₂NH), 3.45 (s, 3H, OCH₃), 2.53 (s, 3H, CH₃), 2.29 (s, 3H, CH₃). ¹³C-NMR (CDCl₃, 75 MHz): δ=169.76 (C═O), 157.90, 154.93, 152.367, 152.23, 150.90, 149.74, 139.34, 134.73, 134.63, 131.16, 130.77, 130.36, 128.85, 129.98, 125.47, 124.66, 123.65, 121.32, 119.51, 119.13, 115.39, 71.96, 59.26, 40.84, 23.57, 16.41.

Using reverse phase high performance liquid chromatography t_{R} (min) was found to be 6.02 for the title compound under the conditions shown in the following table.

| | |
|---|---|
| Symmetry Shield RP18 | 75 × 4.6 mm |
| Flow | 1.0 mL/min |
| Wavelength | 205/210/220/245 nm |
| Temp. | 25° C. |
| Injection Volume | 10 μL of a ca. 0.5% solution in ACN/H₂O 9/1 |
| Eluent B | ACN |
| Eluent C | 0.01 mmol NH₄OAc in H₂O pH = 6.0 |
| Gradient 0 min | B = 30%, C = 70% |
| Gradient 20 min | B = 85%, C = 15% |

### EXAMPLE 4

### Preparation of 2-Methoxy-acetic Acid Propargylamide-9-BBN

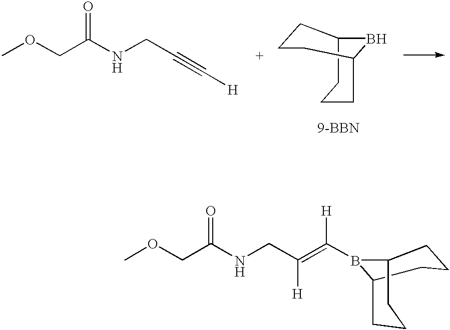

To a solution of 18.88 g of 9-borabicyclononane (9-BBN) in 242 ml of tetrahydrofuran (THF) was added a solution of 19.67 g of 2-methoxy-acetic acid propargylamide and 48.3 ml of THF over 18 minutes at 19° C. to 25° C. The reaction mixture was stirred for 21 hours at ambient temperature.

### EXAMPLE 5

### Preparation of 6-(N-Methoxyacetyl-3-amino-propen-1-yl)-4-[3-methyl-4-(6-methyl-pyridine-3-yloxy)-phenylamino]-quinazoline Using Suzuki Coupling Reaction

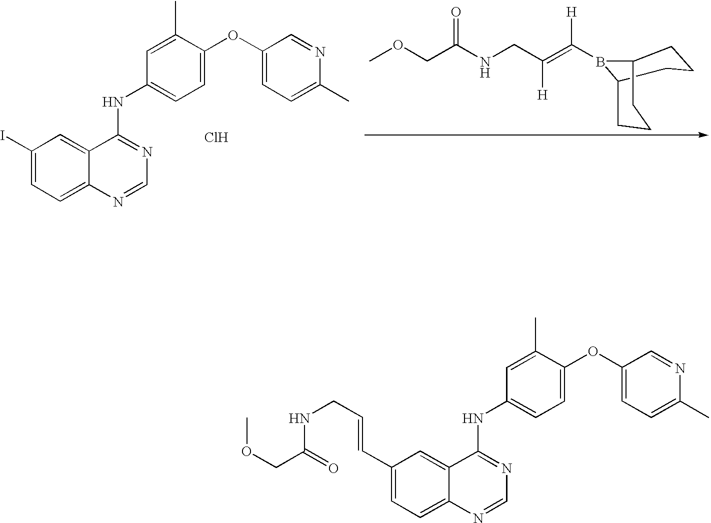

The reaction mixture from Example 4 was cooled to 8.5° C. and a solution of 42.77 g of potassium carbonate in 348 ml of water was added over 42 minutes maintaining the pot temperature between 8° C. and 10° C. To this reaction was added 24.15 g of (6-iodo-quinazolin-4-yl)-[3-methyl-4-(6-methyl-pyridin-3-yloxy)-phenyl]-amine, 541 mg of triphenylphosphine, and 116 mg of palladium (II) acetate and the mixture was heated to 50° C. for 21 hours. The completed reaction was then cooled back to ambient temperature and 290 ml of ethyl acetate (EtOAc) was added and the layers were separated. The product was then extracted from the organic layer into 140 ml of 1 N hydrochloric acid and the product-rich aqueous was washed 4 times with 145 ml of EtOAc. A 1 L round-bottomed flask was charged the product-rich aqueous layer, 290 ml new EtOAc, 2.42 g of KBB Darco (activated carbon, Aldrich, Inc.), 2.42 g filter-aid (Celite, Aldrich, Inc.), and 150 ml of 1N sodium hydroxide. The mixture was stirred for 26 minutes at ambient temperature and then the solids were filtered off through filter-aid and washed with 24 ml of EtOAc. The layers of the filtrate were separated and the product-rich organic layer was washed with 145 ml of water. Next, 140 ml of 30% hydrogen peroxide was charged to the organic layer and stirred for 20 minutes. A solution of 1 g sodium chloride in 4 ml of water was added to the peroxide mixture and the layers were separated. The organic layer was cooled in an ice water bath to 12° C. and a solution of 14.5 g sodium bisulfite in 131 ml of water was added over 23 minutes maintaining the pot temperature less than 26° C. The layers were separated and to the product-rich acidic sodium bisulfite layer was added 300 ml of EtOAc. The pH of the aqueous layer was adjusted to pH to 10-10.5 with 160 ml of 1N sodium hydroxide and the layers were separated. The product-rich organic layer was washed with 100 ml of water, the layers were separated, and the organic layer was vacuum concentrated to 125 ml volume. The concentrate was displaced under vacuum with 483 ml of EtOAc, and the solids were filtered. The solids were washed with 24 ml of EtOAc, and then vacuum dried at 46° C. for 20 hours to provide 10.70 g of light yellow solids. Analytical HPLC assay indicates the solids to be 95.6% 6-(N-Methoxyacetyl-3-amino-propen-1-yl)-4-[3-methyl-4-(6-methyl-pyridine-3-yloxy)-phenylamino]-quinazoline by area percent.

### EXAMPLE 6

### Preparation of 6-(N-Methoxyacetyl-3-amino-propen-1-yl)-4-[3-methyl-4-(6-methyl-pyridine-3-yloxy)-phenylamino]-quinazoline Using Heck Coupling Reaction

To a round bottom flask with nitrogen atmosphere, reflux condenser, oil bath, and overhead stirring was charged 5 vol anhydrous DMF and the solvent was purged with nitrogen. To the flask was added 0.04 eq. palladium acetate, the mixture was purged with nitrogen, then 0.08 eq triphenyl phosphine was added and the mixture was again purged with nitrogen. The mixture was stirred to achieve a red solution (˜30-60 minutes). After the stirring period, the solution was purged with nitrogen and 2 eq sodium acetate, 1 eq (6-Iodo-quinazolin-4-yl)-[3-methyl-4-(6-methyl-pyridin-3-yloxy)-phenyl]-amine and 1.1 eq N-Ally-2-methoxy-acetamide were charged to the flask. The mixture was again purged with nitrogen and heated to 100° C. for 4-6 hours. After reaction completion (as determined by HPLC), the reaction was quenched with 10 vol. 1 N HCl. The aqueous layer was separated and reextracted once with 20 vol. EtOAc. The aqueous layer was basified with 6 N NaOH to pH 9. The product was extracted from the aqueous layer using EtOAC (1×20 vol. and then 1×10 vol.). The organic layer was separated and concentrated to low volume and the remaining ethyl acetate was displaced with acetonitrile. The solution was stirred overnight and the product 6-(N-Methoxyacetyl-3-amino-propen-1-yl)-4-[3-methyl-4-(6-methyl-pyridine-3-yloxy)-phenylamino]-quinazoline precipitated out. The solids were then granulated and filtered.

### EXAMPLE 7

### 6-Chloro-[3-methyl-4-(6-methyl-pyridine-3-yloxy)-phenylamino]-quinazoline

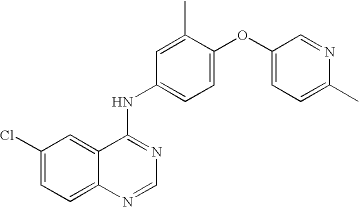

A 3 neck round bottom flask was fitted with a mechanical stirrer and kept under nitrogen. To the flask was charged dry THF (400 ml), dichloroquinazoline (18.6 g, 93.3 mmol) and 3-amino-4-methylpyridine (20.0 g, 93.3 mmol). The light, thin yellow slurry was heated to 50° C. and after approximately 4 hours, the reaction was complete by HPLC. The now darker and much thicker yellow slurry was allowed to slowly cool to 20° C. The desired product was collected by filtration and washed with THF (1×100 ml). The yellow solids were dried at 20° C. with p ˜23 inches Hg. The yellow crystals were obtained in 64% yield (22.41 g). H NMR (CD₃OD, 400 MHz): δ 8.80 (s, 1 H), 8.75 (s, 1 H), 8.38 (s, 1H), 8.12-8.10 (dd, 1 H, J=2.0, 2.4), 7.88-7.83 (m, 2 H), 7.76-7.66 (m, 3 H), 7.17 (d, 1 H, J=8.8), 2.68 (s, 3 H), 2.31 (s, 3 H).

The title compound had a t_{R} (min) of 7.213 under the following RP-HPLC conditions: SB-CN 4.6×150 mm; flow=2 ml/min; λ=237 nm; temp.=20° C.; injection volumes=5 μL; eluent=0.1% phosphoric acid, 0.3% triethylamine in Dl water/MeCN (60:40); gradient at 5 minutes to 90% in 5 minutes and then at 10 m

### EXAMPLE 8

### Preparation of 6-(N-Methoxyacetyl-3-amino-propen-1-yl)-4-[3-methyl-4-(6-methyl-pyridine-3-yloxy)-phenylamino]-quinazoline Using Heck Coupling Reaction

The Heck reaction was carried out by charging a reaction flask with DMF (5 volumes), tri-t-butylphosphine (0.06 equiv.) and palladium acetate (0.04 equiv.). The reaction was charged with nitrogen and allowed to stir for 30 minutes. After stirring, the reaction was charged with 10.0 g (6-chloro-quinazolin-4-yl)-[3-methyl-4-(6-methyl-pyridin-3-yloxy)-phenyl]-amine and 1.1 eq N-Allyl-2-methoxy-acetamide, and sodium acetate (2 equiv.). The reaction was sparged and heated to 100° C. for 24 hours. After cooling, the reaction was deemed complete by HPLC. The work-up was followed by charging 1 N HCl (10 volumes) and ethyl acetate (20 volumes). After separation, the aqueous was washed with 1×20 vol. ethyl acetate. The aqueous was then charged with 0.05 wt equiv. celite, 0.05 wt. equiv. Darco, 10 vol. ethyl acetate, 0.76 vol. THF and 15 volumes 3 N NaOH. The mixture was stirred for 30 minutes and filtered over celite. The layers were separated and the product layer (organic) was washed 1×20 volumes with brine. The product layer was then concentrated to a low volume and any residual water was removed via azeotropic distillation with excess ethyl acetate. The final solution was concentrated to a low volume. The solution was stirred overnight and the product 6-(N-Methoxyacetyl-3-amino-propen-1-yl)-4-[3-methyl-4-(6-methyl-pyridine-3-yloxy)-phenylamino]-quinazoline precipitated out. The solids were then granulated and filtered.

1. A method for preparing a compound of formula 1 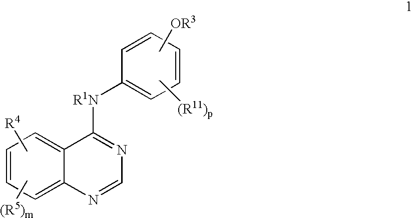
pharmaceutically acceptable salts, solvates and prodrugs thereof, wherein:
m is an integer from 0 to 3;
p is an integer from 0 to 4;
each R¹and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;
R⁴ is —C≡C—(CR¹⁶R¹⁷)ₜR⁹, —C═C—(CR¹⁶R¹⁷)ₜ—R⁹, —C≡C—(CR¹⁶R¹⁷)ₖR¹³, or —C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or —OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R¹⁵;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;
each R¹⁶ and R¹⁷is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally subsituted with a group selected from hydroxy, halo, C₁-C₄ alkyl, C₁-C₄ alkoxy and —NR¹R², which comprises reacting a compound of formula 2 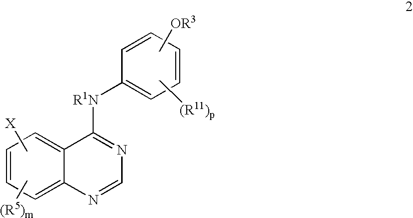
wherein X is a halide and R¹, R³, R⁵, R¹¹, m and p are as defined for formula 1 with a compound of formula H—C≡C—(CR¹⁶R¹⁷)ₜR⁹, M-C═C—(CR¹⁶R¹⁷)ₜ—R⁹, H—C≡C—(CR¹⁶R¹⁷)ₖR¹³, or M-C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3, wherein M is selected from the group consisting of H, B(R¹⁹)₂, Al(R²⁰)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo, wherein R²⁰ is selected from the group consisting of C₁-C₁₀ alkyl, C₁-C₁₀ alkoxy, C₃-C₁₀ cycloalkyl, and halo, wherein R²¹ is C₁-C₁₀ alkyl and wherein W is Cl, Br or I, wherein said reaction is carried out in the presence of a palladium catalyst, a ligand, a base, and an optional additive.

2. A method for preparing a compound of formula 1 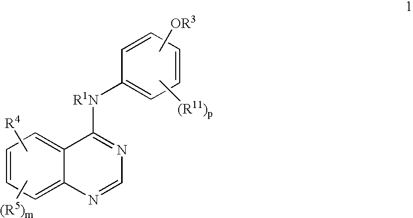
pharmaceutically acceptable salts, solvates and prodrugs thereof, wherein:
m is an integer from 0 to 3;
p is an integer from 0 to 4;
each R¹ and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;
R⁴ is —C≡C—(CR¹⁶R¹⁷)ₜR⁹, —C═C—(CR¹⁶R¹⁷)ₜ—R⁹, —C≡C—(CR¹⁶R¹⁷)ₖR¹³, or —C═C—(CR¹⁶R¹⁷)ₖR¹³, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or 13 OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R¹⁵;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;
each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally subsituted with a group selected from hydroxy, halo, C₁-C₄ alkyl, C₁-C₄ alkoxy and —NR¹R², which comprises reacting a compound of formula 3 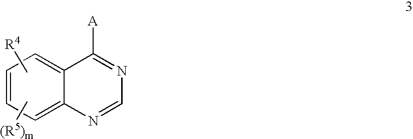
wherein A is Cl or F and R⁴, R⁵ and m are as defined for formula 1 with a compound of formula 4 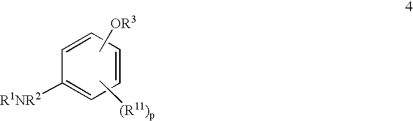
wherein R¹, R², R³, R¹¹ and p are as defined for formula 1.

3. The method according to claims 1 or 2, wherein R³ is —(CR¹R²)ₜ(4 to 10 membered heterocyclic), wherein t is an integer from 0 to 5, and the foregoing R³ groups are optionally substituted by 1 to 3 R⁸ groups.

4. The method according to claim 3, wherein said heterocyclic group is optionally fused to a benzene ring or a C₅-C₈ cycloalkyl group, and the foregoing R³ groups, including any optional fused rings referred to above, are optionally substituted by 1 to 3 R⁸ groups.

5. The method according to claims 1 or 2, wherein R³ is selected from 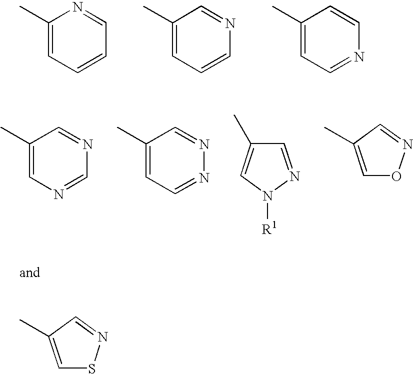
wherein the foregoing R³ groups are optionally substituted by 1 to 3 R⁸ groups.

6. The method according to claims 1 or 2, wherein R³ is pyridin-3-yl optionally substituted by 1 to 3 R⁸ groups.

7. The method according to claims 1 or 2, wherein R⁴ is —C≡C—(CR¹⁶R¹⁷)ₜR⁹, wherein m is an integer from 0 to 3, and t is an integer from 0 to 5.

8. The method according to claims 1 or 2, wherein R⁴ is —C≡C—(CR¹⁶R¹⁷)ₜR⁹, wherein m is an integer from 0 to 3, and t is an integer from 0 to 5, wherein R⁹ is selected from 3-piperidinyl and 4-piperidinyl each of which is optionally substituted with 1 or 2 R⁸ groups.

9. The method according to claims 1 or 2, wherein R⁴ is —C═C—(CR¹⁶R¹⁷)ₜ—R⁹, wherein m is an integer from 0 to 3, and t is an integer from 0 to 5.

10. The method according to claims 1 or 2, wherein R⁴ is —C═C—(CR¹⁶R¹⁷)ₜ—R⁹, wherein m is an integer from 0 to 3, and t is an integer from 0 to 5, wherein R⁹ is selected from 3-piperidinyl and 4-piperidinyl (optionally substituted with 1 or 2 R⁸ groups).

11. The method according to claims 1 or 2, wherein R⁴ is —C≡C—(CR¹⁶R¹⁷)ₖR¹³, wherein k is an integer from 1 to 3 and m is an integer from 0 to 3.

12. The method according to claims 1 or 2, wherein R⁴ is —C≡C—(CR¹⁶R¹⁷)ₖR¹³, wherein k is an integer from 1 to 3 and m is an integer from 0 to 3, wherein R¹³ is —NR¹R¹⁴, wherein R¹⁴ is selected from —C(O)R¹⁵, —SO₂R¹⁵, and —C(O)NR¹⁵R⁷.

13. The method according to claims 1 or 2, wherein R⁴ is —C═C—(CR¹⁶R¹⁷)ₖR¹³, wherein k is an integer from 1 to 3 and m is an integer from 0 to 3.

14. The method according to claims 1 or 2, wherein R⁴ is —C═C—(CR¹⁶R¹⁷)ₖR¹³, wherein k is an integer from 1 to 3 and m is an integer from 0 to 3, wherein R¹³ is —NR¹R¹⁴, wherein R¹⁴ is selected from —C(O)R¹⁵, —SO₂R¹⁵, and —C(O)NR¹⁵R⁷.

15. The method according to claims 1 or 2, wherein R⁴ is —C≡C—(CR¹⁶R¹⁷)ₖR¹³ or —C═C—(CR¹⁶R¹⁷)ₖR¹³, wherein k is an integer from 1 to 3 and m is an integer from 0 to 3, R¹³ is —NR¹R¹⁴ or —OR¹⁴, R¹⁴ is R¹⁵, R¹⁵ is R¹⁸, and R¹⁸ is C₁-C₆ alkyl optionally substituted by —OR⁶, —S(O)ⱼR⁶, —NR⁶R⁷, —NR⁶C(O)R⁷, —NR⁶SO₂R⁷, —NR⁶CO₂R⁷, CN, —C(O)R⁶, or halo.

16. The method according to claims 1 or 2, wherein the compound prepared is selected from the group consisting of:
(±)-[3-Methyl-4-(pyridin-3-yloxy)-phenyl]-(6-piperidin-3-ylethynyl-quinazolin-4-yl)-amine;
2-Methoxy-N-(3-{4-[3-methyl-4-(pyridin-3-yloxy)-phenylamino]-quinazolin-6-yl}-prop-2-ynyl)-acetamide
(±)-[3-Methyl-4-(6-methyl-pyridin-3-yloxy)-phenyl]-(6-piperidin-3-ylethynyl-quinazolin-4-yl)-amine;
[3-Methyl-4-(6-methyl-pyridin-3-yloxy)-phenyl]-(6-piperidin-4-ylethynyl-quinazolin-4-yl)-amine;
2-Methoxy-N-(3-{4-[3-methyl-4-(6-methyl-pyridin-3-yloxy)-phenylamino]-quinazolin-6-yl}-prop-2-ynyl)-acetamide;
2-Fluoro-N-(3-{4-[3-methyl-4-(6-methyl-pyridin-3-yloxy)-phenylamino]-quinazolin-6-yl}-prop-2-ynyl)-acetamide;
E-2-Methoxy-N-(3-{4-[3-methyl-4-(6-methyl-pyridin-3-yloxy)-phenylamino]-quinazolin-6-yl}-allyl)-acetamide;
[3-Methyl-4-(pyridin-3-yloxy)-phenyl]-(6-piperidin-4-ylethynyl-quinazolin-4-yl)-amine;
2-Methoxy-N-(1-{4-[3-methyl-4-(6-methyl-pyridin-3-yloxy)-phenylamino]-quinazolin-6-ylethynyl}-cyclopropyl)-acetamide;
E-N-(3-{4-[3-Chloro-4-(6-methyl-pyridin-3-yloxy)-phenylamino]-quinazolin-6-yl}-allyl)-2-methoxy-acetamide;
N-(3-{4-[3-Chloro-4-(6-methyl-pyridin-3-yloxy)-phenylamino]-quinazolin-6-yl}-prop-2-ynyl)-acetamide;
N-(3-{4-[3-Methyl-4-(6-methyl-pyridin-3-yloxy)-phenylamino]-quinazolin-6-yl}-prop-2-ynyl)-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
1-Ethyl-3-(3-{4-[3-methyl-4-(6-methyl-pyridin-3-yloxy)-phenylamino]-quinazolin-6-yl}-prop-2-ynyl)-urea;
Piperazine-1-carboxylic acid (3-{4-[3-methyl-4-(6-methyl-pyridin-3-yloxy)-phenylamino]-quinazolin-6-yl}-prop-2-ynyl)-amide;
(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;
2-Dimethylamino-N-(3-{4-[3-methyl-4-(pyridin-3-yloxy)-phenylamino]-quinazolin-6-yl}-prop-2-ynyl)-acetamide;
E-N-(3-{4-[3-Methyl-4-(6-methyl-pyridin-3-yloxy)-phenylamino]-quinazolin-6-yl}-allyl)-methanesulfonamide;
Isoxazole-5-carboxylic acid (3-{4-[3-methyl-4-(6-methyl-pyridin-3-yloxy)-phenylamino]-quinazolin-6-yl}-prop-2-ynyl)-amide;
1-(1,1-Dimethyl-3-{4-[3-methyl-4-(6-methyl-pyridin-3-yloxy)-phenylamino]-quinazolin-6-yl}-prop-2-ynyl)-3-ethyl-urea;
and the pharmaceutically acceptable salts, prodrugs and solvates of the foregoing compounds.

17. The method according to claim 1, wherein the compound of formula 2 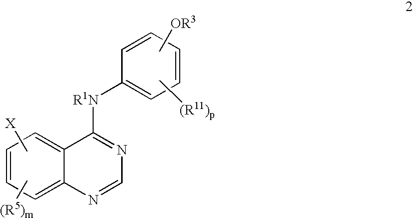
is prepared by reacting a compound of formula 2A 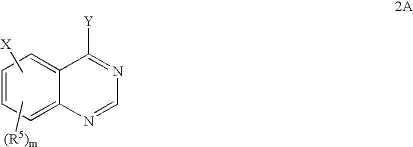
wherein Y is a halide and X, R⁵ and m are as defined for formula 1, with a compound of formula E 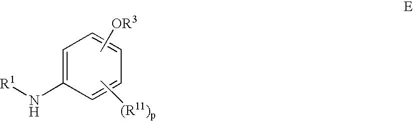
wherein R¹, R³, R¹¹, and p are as defined for formula 1.

18. The method according to claim 1, wherein X is Br or I, R⁴ is —C═C—(CR¹⁶R¹⁷)ₜ—R⁹, or —C═C—(CR¹⁶R¹⁷)ₖR¹³ and said reaction is carried out in the presence of a palladium or nickel catalyst selected from the group consisting of Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, BnPdCl(PPh₃)₂, Pd(Otfa)₂, Pd(PPh₃)₂(Otfa)₂, PdCl₂(dppf), Pd(acac)₂, Pd₂(dba)₃-CHCl₃, Ni(PPh₃)₄, Pd(dppb),

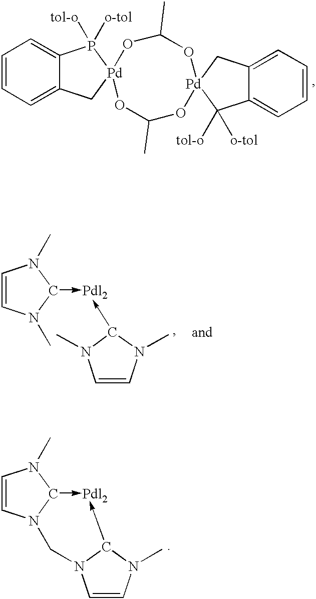

19. The method according to claim 18, wherein said palladium catalyst is selected from the group consisting of Pd(OAc)₂, Pd₂(dba)₃, and Pd(PPh₃)₄.

20. The method according to claim 19, wherein said palladium catalyst is selected from the group consisting of Pd(OAc)₂ and Pd(PPh₃)₄.

21. The method according to claim 1, wherein said ligand is selected from the group consisting of a polymer bound phosphine, 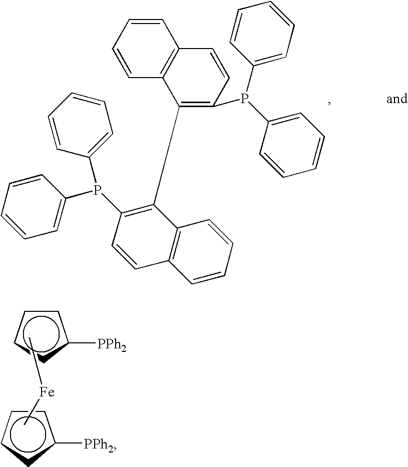
2-methyl-2′-(dicyclohexylphosphino)biphenyl, 2-dimethylamino-2′-(dicyclohexylphosphino)biphenyl, and P(R²²)₃, wherein each R²² is independently selected from the group consisting of 2-methyl-2′-(dicyclohexylphosphino)biphenyl, 2-dimethylamino-2′-(dicyclohexylphosphino)biphenyl, phenyl, o-toluyl, OMe, and furyl,

22. The method according to claim 21, wherein said ligand is selected from the group consisting of PPh₃, P(o-Tol)₃, P(o-OMePh)₃, P(2-Furyl)₃,

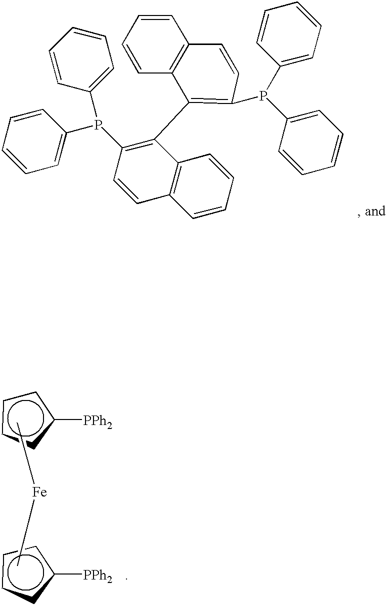

23. The method according to claim 22, wherein said ligand is selected from the group consisting of PPh₃, P(o-Tol)₃, and P(2-Furyl)₃.

24. The method according to claim 1, wherein M is selected from the group consisting of H, Al(R²⁰)₂, Sn(R²¹)₃, MgW, and ZnW and wherein said base is selected from the group consisting of (R)₃N, (R)₂NH, RNH₂, QX, Q₂CO₃, Q₃PO₄, QO₂CR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

25. The method according to claim 24, wherein said base is selected from the group consisting of R₄NF, R₄NCl, R₄NBr, Et₃N, Me₂NEt, iPr₂NEt, CuBr, CuI, CdCl, CsF, K₂CO₃, Na₃PO₄, Na₂HPO₄, NaOAc, DABCO, and 1,8-(dimethylamino)napthalene, wherein each R is independently selected from the group consisting of C₁-C₆ alkyl, phenyl and benzyl.

26. The method according to claim 25, wherein said base is selected from the group consisting of Et₃N, Me₂NEt, K₂CO₃, Na₃PO₄ and NaOAc.

27. The method according to claim 1, wherein said reaction is carried out in a solvent selected from the group consisting of toluene, benzene, xylene, dimethylformamide, dimethylacetamide, dioxane, tetrahydrofuran, acetonitrile, N-methylpyrrolidinone, dimethylsulfoxide, dimethoxyethane, CH₂Cl₂, CHCl₃, ClCH₂CH₂Cl, N(C₁-C₆ alkyl)₃, N(benzyl)₃, and mixtures thereof.

28. The method according to claim 27, wherein said solvent selected from the group consisting of toluene, dimethylformamide, dimethylacetamide, dioxane, tetrahydrofuran, acetonitrile, N-methylpyrrolidinone, dimethoxyethane, ClCH₂CH₂Cl, N(C₁-C₆ alkyl)₃, N(benzyl)₃ and mixtures thereof.

29. The method according to claim 28, wherein the solvent is selected from tetrahydrofuran, dioxane, dimethoxyethane, dimethylformamide, dimethylacetamide, and mixtures thereof.

30. The method according to claim 1, wherein said reaction is carried out at a temperature ranging from about 25° C. to about 175° C.

31. The method according to claim 1, wherein M is B(R¹⁹)₂ and wherein said base is selected from the group consisting of (R)₃N, (R)₂NH, RNH₂, DABCO, 1,8-(dimethylamino)napthalene, QX, Q₂CO₃, Q₃PO₄, Q₂HPO₄, QO₂CR, QOH, and QOR, wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

32. The method according to claim 31, wherein said base is selected from the group consisting of NaOEt, NaOMe, NaOH, KOH, LiOH, Ca(OH)₂, TIOH, Ba(OH)₂, Et₃N, Me₂NEt, iPr₂NEt, CuBr, CuI, CdCl, CsF, KF, KCl, K₂CO₃, Na₃PO₄, Na₂HPO₄, NaOAc, DABCO, 1,8-(dimethylamino)napthalene, R₄NF, R₄NCl, and R₄NBr, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

33. The method according to claim 18, wherein said solvent is selected from the group consisting of toluene, benzene, xylene, dimethylformamide, dimethylacetamide, water, dioxane, tetrahydrofuran, acetonitrile, N-methylpyrrolidinone, dimethylsulfoxide, dimethoxyethane, acetone, CH₂Cl₂, CHCl₃, ClCH₂CH₂Cl, N(R)₃ and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

34. The method according to claim 33, wherein said solvent selected from the group consisting of toluene, dimethylformamide, dimethylacetamide, water, tetrahydrofuran, N-methylpyrrolidinone, methanol, ethanol, isopropanol, dimethoxyethane, ClCH₂CH₂Cl, N(R)₃, and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

35. The method according to claim 34, wherein said solvent is tetrahydrofuran, water, or a mixture of tetrahydrofuran and water.

36. The method according to claim 18, wherein said reaction is carried out at a temperature ranging from about 25° C. to about 175° C.

37. The method according to claim 1, wherein the compound of formula 1 is prepared by reacting a compound of formula 2 with a compound of formula H—C≡C—(CR¹⁶R¹⁷)ₖR¹³ wherein —(CR¹⁶R¹⁷)ₖR¹³ is selected from the group consisting of —CH₂NHC(O)R¹⁵, —CH₂NHSO₂R¹⁵, and —CH₂NHCO₂R¹⁵ wherein said HC≡CCH₂NHC(O)R¹⁵, HC≡CCH₂NHSO₂R¹⁵, and HC≡CCH₂NHCO₂R¹⁵ are prepared by reacting HC≡CCH₂NH₂ with a compound of formula ClC(O)R¹⁵, ClSO₂R¹⁵, or ClCO₂R¹⁵, respectively.

38. The method according to claim 1, wherein the compound of formula 1 is prepared by reacting a compound of formula 2 with a compound of formula M-C═C—(CR¹⁶R¹⁷)ₖR¹³ wherein —(CR¹⁶R¹⁷)ₖR¹³ is selected from the group consisting of —CH₂NHC(O)R¹⁵, —CH₂NHSO₂R¹⁵, and —CH₂NHCO₂R¹⁵ wherein said H₂C═CCH₂NHC(O)R¹⁵, H₂C═CCH₂NHSO₂R¹⁵ and H₂C═CCH₂NHCO₂R¹⁵ are prepared by reacting HC═CCH₂NH₂ with a compound of formula ClC(O)R¹⁵, ClSO₂R¹⁵, or ClCO₂R¹⁵, respectively.

39. The method according to claim 1, wherein the compound of formula 3 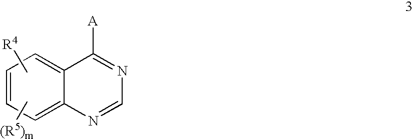
is prepared by reacting a compound of formula 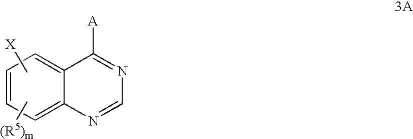
wherein X is a halide, A, R⁵, and m are as defined in claim 1 with a compound of formula MR⁴, wherein R⁴ and M are as defined in claim 1.

40. The method according to claim 39, wherein X is Br or I, and wherein R⁴ is —C═C—(CR¹⁶R¹⁷)ₜ—R⁹, or —C═C—(CR¹⁶R¹⁷)ₖR¹³ and said reaction is carried out in the presence of a palladium or nickel catalyst selected from the group consisting of Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, BnPdCl(PPh₃)₂, Pd(Otfa)₂, Pd(PPh₃)₂(Otfa)₂, PdCl₂(dppf), Pd(acac)₂, Pd₂(dba)₃-CHCl₃, Ni(PPh₃)₄, Pd(dppb),

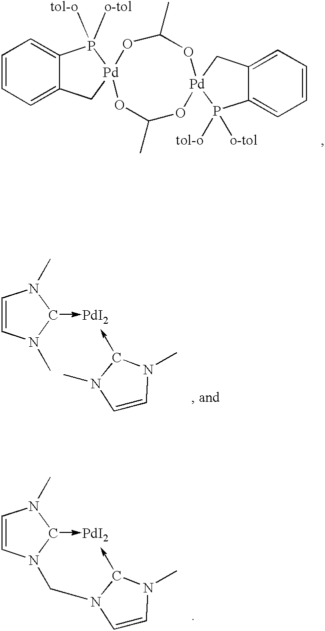

41. The method according to claim 40, wherein said palladium catalyst is selected from the group consisting of Pd(OAc)₂, Pd₂(dba)₃, and Pd(PPh₃)₄.

42. The method according to claim 41, wherein said palladium catalyst is selected from the group consisting of Pd(OAc)₂ and Pd(PPh₃)₄.

43. The method according to claims 18, wherein said ligand is selected from the group consisting of a polymer bound phosphine, 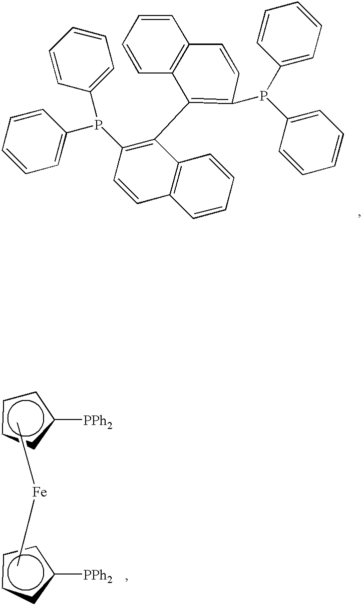
2-methyl-2′-(dicyclohexylphosphino)biphenyl, 2-dimethylamino-2′-(dicyclohexylphosphino)biphenyl, and P(R²²)₃, wherein each R²² is independently selected from the group consisting of 2-methyl-2′-(dicyclohexylphosphino)biphenyl or 2-dimethylamino-2′-(dicyclohexylphosphino)biphenyl, phenyl, o-toluyl, OMe, and furyl.

44. The method according to claim 43, wherein said ligand is selected from the group consisting of PPh₃, P(o-Tol)₃, P(o-OMePh)₃, P(2-Furyl)₃,

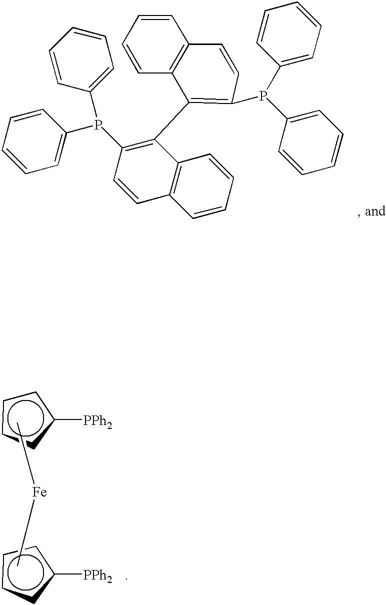

45. The method according to claim 44, wherein said ligand is selected from the group consisting of PPh₃, P(o-Tol)₃, and P(o-Furyl)₃.

46. The method according to claim 18, wherein said solvent is selected from the group consisting of toluene, benzene, xylene, dimethylformamide, dimethylacetamide, water, dioxane, tetrahydrofuran, acetonitrile, N-methylpyrrolidinone, dimethylsulfoxide, dimethoxyethane, acetone, CH₂Cl₂, CHCl₃, ClCH₂CH₂Cl, N(R)₃, and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

47. The method according to claim 46, wherein said solvent selected from the group consisting of toluene, dimethylformamide, dimethylacetamide, dioxane, tetrahydrofuran, acetonitrile, N-methylpyrrolidinone, dimethoxyethane, ClCH₂CH₂Cl, and N(R)₃, and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁₁-C₆ alkoxy, and wherein R¹ and R² are as defined for formula 1.

48. The method according to claim 47, wherein solvent is selected from the tetrahydrofuran, dioxane, dimethoxyethane, dimethylformamide, dimethylacetamide, and mixtures thereof.

49 The method according to claim 18, wherein said base is selected from the group consisting of Et₃N, Me₂NEt, iPr₂NEt, CuBr, CuI, CdCl, CsF, R₄NF, R₄NCl, R₄NBr, K₂CO₃, Na₃PO₄, Na₂HPO₄, NaOAc, DABCO, and 1,8-(dimethylamino)napthalene, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

50. The method according to claim 49, wherein said base is selected from the group consisting of Et₃N, Me₂NEt, K₂CO₃, Na₃PO₄ and NaOAc.

51. The method according to claim 18, wherein said reaction is carried out at a temperature ranging from about 25° C. to about 175° C.

52. The method according to claim 1, wherein M is B(R¹⁹)₂ and wherein said base is selected from the group consisting of (R)₃N, (R)₂NH, RNH₂, DABCO, 1,8-(dimethylamino)napthalene, QX, Q₂CO₃, Q₃PO₄, Q₂HPO₄, QO₂CR, QOH, and QOR wherein Q is selected from the group consisting of (R)₄N, Na, K, Cs, Cu, Cd, and Ca, and wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

53. The method according to claim 52, wherein said base is selected from the group consisting of NaOEt, NaOMe, NaOH, KOH, LiOH, Ca(OH)₂, TIOH, Ba(OH)₂, Et₃N, Me₂NEt, iPr₂NEt, CuBr, CuI, CdCl, CsF, KF, KCl, K₂CO₃, Na₃PO₄, Na₂HPO₄, NaOAc, DABCO, 1,8-(dimethylamino)napthalene, R₄NF, R₄NCl, and R₄NBr, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

54. The method according to claim 52, wherein said solvent is selected from the group consisting of toluene, benzene, xylene, dimethylformamide, dimethylacetamide, water, dioxane, tetrahydrofuran, acetonitrile, N-methylpyrrolidinone, dimethylsulfoxide, dimethoxyethane, acetone, CH₂Cl₂, CHCl₃, ClCH₂CH₂Cl, N(R)₃, and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

55. The method according to claim 54, wherein said solvent selected from the group consisting of toluene, dimethylformamide, dimethylacetamide, water, tetrahydrofuran, N-methylpyrrolidinone, methanol, ethanol, isopropanol, dimethoxyethane, ClCH₂CH₂Cl, N(R)₃ and mixtures thereof, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1.

56. The method according to claim 55, wherein said solvent is tetrahydrofuran, water or a mixture of tetrahydrofuran and water.

57. The method according to claim 52, wherein said reaction is carried out at a temperature ranging from about 25° C. to about 175° C.

58. The method according to claim 1, wherein X is Cl, wherein R⁴ is —C═C—(CR¹⁶ R¹⁷)ₜ—R⁹, or —C═C—(CR¹⁶R¹⁷)ₖR¹³, wherein M is B(R¹⁹)₂, and said reaction is carried out in the presence of a catalyst, ligand, base, and solvent, wherein catalyst, ligand, base, and solvent is selected from one of the following groups:
(vi) said catalyst is Pd₂(dba)₃ or Pd(OAc)₂, said ligand is 2-methyl-2′-(dicyclohexylphosphino)biphenyl, 2-dimethylamino-2′-(dicyclohexylphosphino)biphenyl, and P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of (R)₄N, M₂CO₃, M₃PO₄, and MX, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1, wherein M is selected from the group consisting of Na, K, Cs and X is halide, and said solvent is selected from the group consisting of toluene, benzene, xylene, DME, acetone, dioxane, DMF, DMAC, NMP, and ACN; or
(vii) said catalyst is selected from the group consisting of Pd(OAc)₂, PdCl₂, Pd(MeCN)₂Cl₂, Pd(PhCN)₂Cl₂, and PdCl₂(PPh₃)₂, said ligand is Ph₄PX, wherein X is selected from the group consisting of Cl, Br, and I, said base is NaOAc or NN dimethylglycine, and said solvent is selected from the group consisting of DMF, DMAC, water, dioxane, THF, ACN, and NMP; or
(viii) said catalyst is selected from the group consisting of Pd(OAc)₂, PdCl₂, Pd(MeCN)₂Cl₂, and Pd(PhCN)₂Cl₂, said ligand is P(OR)₃, wherein R is selected from the group consisting of Et, iPr, Ph, 2,4-dit-BuPh, and Ar, said base is selected from the group consisting of (R)₄N, M₂CO₃ and MO₂CR, wherein M is selected from the group consisting of Na, K, and Cs, where each R is independently selected from C₁-C₆ alkyl or benzyl, and said solvent is selected from the group consisting of DMF, DMAC, water, dioxane, THF, ACN, and NMP; or
(ix) said catalyst is selected from the group consisting of 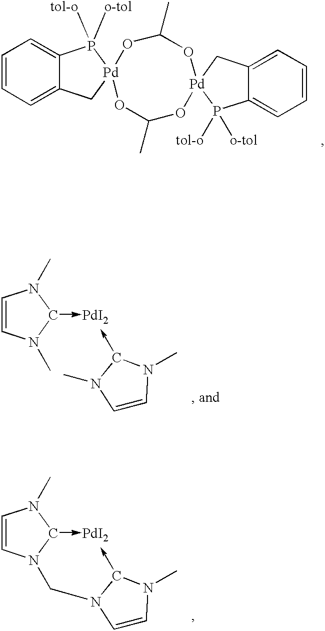
wherein said ligand is P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is (R)₄N or M₂CO₃, wherein M is selected from the group consisting of Na, K, and Cs, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, DME, acetone, Dioxane, DMF, DMAC, and NMP; and
(x) said catalyst is Pd₂(dba)₃, said ligand is Ligand 4 or 5, said base is (R)₄N or M₂CO₃, wherein M is selected from the group consisting of Na, K, and Cs, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R² trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, DME, acetone, Dioxane, DMF, DMAC, and NMP.

59. The method according to claim 1, wherein X is chlorine, R⁴ is —C═C—(CR¹⁶R¹⁷)ₜ—R⁹, or —C═C—(CR¹⁶R¹⁷)ₖR¹³ and M is H and said reaction is carried out in the presence of a catalyst, ligand, base, and solvent mixture comprised of one of the following:
(i) said catalyst is Pd₂(dba)₃ or Pd(OAc)₂, said ligand is 2-methyl-2′-(dicyclohexylphosphino)biphenyl, 2-dimethylamino-2′-(dicyclohexylphosphino)biphenyl, and P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of M₂CO₃, M₃PO₄, and MX wherein M is selected from the group consisting of Na, K, Cs, and (R)₄N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, DME, acetone, Dioxane, DMF, DMAC, NMP, and ACN; or
(ii) said catalyst is selected from the group consisting of Pd(OAc)₂, PdCl₂, Pd(MeCN)₂Cl₂, Pd(PhCN)₂Cl₂, and PdCl₂(PPh₃)₂, said ligand is Ph₄PX, wherein X is selected from the group consisting of Cl, Br, and I, said base is NaOAc or NN dimethylglycine, and said solvent is selected from the group consisting DMF, DMAC, water, dioxane, THF, ACN, and NMP; or
(iii) said catalyst is selected from the group consisting of 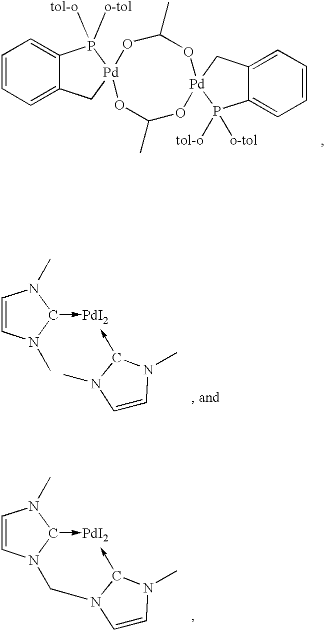
said base is NaOAc, Bu₄NBr, hydrazine, or NaOCHO, and said solvent is selected from the group consisting toluene, benzene, xylene, DME, acetone, Dioxane, DMF, DMAC, and NMP; and
(iv) said catalyst is Pd₂(dba)₃, said ligand is 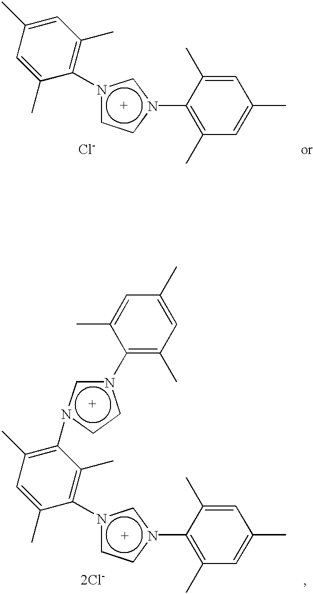
said base is selected from the group consisting NaOAc, Bu₄NBr, hydrazine, and NaOCHO and said solvent is selected from the group consisting toluene, benzene, xylene, DME, acetone, dioxane, DMF, DMAC, and NMP.

60. The method according to claim 1, wherein X is chlorine, R⁴ is —C═C—(CR¹⁶R¹⁷)ₜ—R⁹, or —C═C—(CR¹⁶R¹⁷)ₖR¹³ and M is Sn(R)₃, said reaction is carried out in the presence of a catalyst, ligand, base, and solvent mixture, wherein said catalyst is Pd₂(dba)₃ or Pd(OAc)₂, said ligand is P(R²²)₃, wherein R²² is selected from the group consisting of C₁-C₆ alkyl, 2-methyl-2′-(dicyclohexylphosphino)biphenyl and 2-dimethylamino-2′-(dicyclohexylphosphino)biphenyl, said base is selected from the group consisting of M₂CO₃, M₃PO₄, MOH and MX, wherein M is selected from the group consisting of Na, K, Cs, and (R)₄N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of DME DMF, DMAC, water, dioxane, THF, ACN, and NMP.

61. The method according to claims 1 and 39, wherein X is Br or I, R⁴ is —C≡C—(CR¹⁶R¹⁷)ₜR⁹ or —C≡C—(CR¹⁶R¹⁷)ₖR¹³, said reaction is carried out in the presence of a catalyst, ligand, base, and solvent mixture, wherein said catalyst is selected from, the group consisting of Pd(OAc)₂, Pd₂(dba)₃, PdCl₂, Pd(MeCN)₂Cl₂, Pd(PhCN)₂Cl₂, PdCl₂(PPh₃)₂, Pd(PPh₃)₄, Pd(Otfa)₂, Pd(PPh₃)₂(Otfa)₂, PdCl₂(dppf), Pd(acac)₂, Pd₂(dba)₃-CHCl₃, and Pd(dppb), said ligand is selected from the group consisting of PPh₃, P(o-Tol)₃, P(o-OMePh)₃, P(2-Furyl)₃, said base is selected from the group consisting of (R)₂NH, RNH₂, and (R)₃N, wherein each R is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, and C₁-C₆ alkoxy, and wherein R¹ and R² are as defined for formula 1 and said solvent is selected from the group consisting of toluene, benzene, xylene, dimethylformamide, dimethylacetamide, dioxane, tetrahydrofuran, acetonitrile, N-methylpyrrolidinone, dimethoxyethane, acetone, CH₂Cl₂, CHCl₃, and ClCH₂CH₂Cl.

62. A compound of formula H—C≡C—(CR¹⁶R¹⁷)ₜR⁹, wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
wherein R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O) moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups, wherein each R¹ and R² is independently selected from H and C₁-C₆ alkyl, wherein each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)j(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5; each R⁶ and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy; or R⁶ and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, and R⁷ are attached, selected from N, N(R¹), O, and S, provided two O atoms, two S atoms or an O and S atom are not attached directly to each other; and t is an integer from 0 to 5.

63. A compound of formula M-C═C—(CR¹⁶R¹⁷)ₜ—R⁹, wherein M is selected from the group consisting of H, B(R¹⁹)₂, Al(R²⁰)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo wherein R²⁰ is selected from the group consisting of C₁-C₁₀ alkyl, C₁-C₁₀ alkoxy, C₃-C₁₀ cycloalkyl, and halo, wherein R is C₁-C₁₀ alkyl and wherein W is Cl, Br or I;
wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
wherein R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups, wherein each R¹ and R² is independently selected from H and C₁-C₆ alkyl, wherein each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷ —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5; each R⁶, and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl C₂-C₆ alkenyl, C₂-C₆ alkynyl, hydroxy, and C₁-C₆ alkoxy; or R⁶ and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, and R⁷ are attached, selected from N, N(R¹), O, and S, provided two O atoms, two S atoms or an O and S atom are not attached directly to each other; and t is an integer from 0 to 5.

64. A compound of formula H—C≡C—(CR¹⁶R¹⁷)ₖR¹³, wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
wherein R¹³ is —NR¹R¹⁴ or —OR¹⁴, wherein R¹⁴ is H, R¹⁵, —C(O)R¹⁵, —SO₂R¹⁵, —C(O)NR¹⁵R⁷, —SO₂NR¹⁵R⁷, or —CO₂R¹⁵, wherein R¹⁵ is R¹⁸, —(CR¹R²)ₜ(C₆-C₁₀ aryl), —(CR¹R²),(4 to 10 membered heterocyclic), wherein each R¹ and R² is independently selected from H and C₁-C₆ alkyl, wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents, wherein R¹⁸ is C₁-C₆ alkyl wherein each carbon not bound to a N or O atom, or to S(O)ⱼ, wherein j is an integer from 0 to 2, is optionally substituted with R¹², wherein R¹² is R⁶, OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2, each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), and wherein t is an integer from 0 to 5.

65. A compound of formula M-C═C—(CR¹⁶R¹⁷)ₖR¹³, wherein M is selected from the group consisting of H, B(R¹⁹)₂, Al(R²⁰)₂, Sn(R²¹)₃, MgW, or ZnW, wherein R¹⁹ is selected from the group consisting of 9-BBN, C₁-C₁₀ alkyl, C₁-C₁₀ alkoxy, C₃-C₁₀ cycloalkyl, and halo wherein R²⁰ is selected from the group consisting of C₁-C₁₀ alkyl, C₁-C₁₀ alkoxy, C₃-C₁₀ cycloalkyl, and halo, and wherein R²¹ is C₁-C₁₀ alkyl and: wherein W is Cl, Br or I;
wherein each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
wherein R¹³ is —NR¹R¹⁴ or —OR¹⁴, wherein R¹⁴ is H, R¹⁵, —C(O)R¹⁵, —SO₂R¹⁵, —C(O)NR¹⁵R⁷, —SO₂NR¹⁵R⁷, or —CO₂R¹⁵, wherein R¹⁵ is R¹⁸, —(CR¹R²)ₜ(C₆-C₁₀ aryl), —(CR¹R²)ₜ(4 to 10 membered heterocyclic), wherein each R¹ and R² is independently selected from H and C₁-C₆ alkyl, wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents, wherein R¹⁸ is C₁-C₆ alkyl wherein each carbon not bound to a N or 0 atom, or to S(O)ⱼ, wherein j is an integer from 0 to 2, is optionally substituted with R¹², wherein R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2, each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)N R⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ⱼ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), and wherein t is an integer from 0 to 5.
